(19) United States
(12) Patent Application Publication
(10) Pub. No.: US 2024/0053359 A1
(43) Pub. Date: Feb. 15, 2024

(54) NATIVE MICROFLUIDIC CE-MS ANALYSIS OF ANTIBODY CHARGE HETEROGENEITY

(71) Applicant: Regeneron Pharmaceuticals, Inc., Tarrytown, NY (US)

(72) Inventors: Hongxia Wang, Briarcliff Manor, NY (US); Haibo Qiu, Hartsdale, NY (US); Ning Li, New Canaan, CT (US); Zhijie Wu, Stamford, CT (US)

(21) Appl. No.: 18/204,801

(22) Filed: Jun. 1, 2023

Related U.S. Application Data
(63) Continuation-in-part of application No. 17/870,368, filed on Jul. 21, 2022.
Application No. 17/870,368 is a continuation-in-part of application No. 16/777,230, filed on Jan. 30, 2020.
(60) Provisional application No. 62/799,331, filed on Jan. 31, 2019.
(60) Provisional application No. 62/851,365, filed on May 22, 2019.

(51) Int. Cl. G01N 33/68

(52) U.S. Cl. CPC G01N 33/6854; G01N 33/6851; G01N 2440/00; G01N 2333/98

(57) ABSTRACT

The present invention pertains to methods for characterizing proteins in a sample using native capillary electrophoresis-mass spectrometry. The present invention pertains to methods for detecting and/or discriminating between post-translational modification variants of an antibody of interest in a sample, detecting and/or discriminating between antibodies in an antibody mixture, and characterizing monospecific antibody side products in a bispecific antibody sample.

### CROSS-REFERENCE TO RELATED APPLICATIONS

This application is a continuation-in-part of U.S. patent application Ser. No. 17/870,368, filed on Jul. 21, 2022, which is continuation-in-part of U.S. application Ser. No. 16/777,230, filed Jan. 30, 2020, which claims the benefit of U.S. Provisional Application No. 62/851,365, filed May 22, 2019, and U.S. Provisional Application No. 62/799,331, filed Jan. 31, 2019, each of which is incorporated by reference in its entirety.

### REFERENCE TO A SEQUENCE LISTING

The instant application contains a Sequence Listing which has been filed electronically in XML format and is hereby incorporated by reference in its entirety. Said XML copy, created on Oct. 14, 2022, is named 070816-02093_SL.xml and is 2,325 bytes in size.

### FIELD OF THE INVENTION

The present invention pertains to biopharmaceuticals, and relates to the use of capillary electrophoresis and mass spectral analysis to characterize size and charge variants of therapeutic antibodies.

### BACKGROUND

Therapeutic antibodies are a significant class of biotherapeutic products, and they have achieved outstanding success in treating many life-threatening and chronic diseases. However, in certain circumstances therapeutic antibodies, such as monoclonal antibodies (mAbs), are heterogeneous molecules produced in mammalian cells with many product variants, including variants resulting from post-translational modifications (PTMs). Variants produced via PTMs can occur throughout the lifespan of a mAb during production, purification, storage, and post-administration. These variants or product-related modifications are also referred to as product quality attributes (PQAs). Controlling PQAs within predefined acceptance criteria is vital to the biopharmaceutical industry because it ensures consistent product quality and reduces potential impacts on drug safety and efficacy.

Each individual monoclonal antibody may therefore present a unique profile, a characteristic which needs to be taken into consideration during the evaluation of these products both during development and manufacturing of final product. A Food and Drug Administration guidance for industry recommends that sponsors should evaluate susceptibilities of therapeutic proteins to modifications within the in vivo milieu (see, Guidance for Industry, Immunogenicity Assessment for Therapeutic Protein Products. 2014). As a result, in vitro and/or in vivo behavior of many PQAs, including deamidation (see, for example, Huang et al., Analytical chemistry 2005; 77:1432-9; Ouellette et al., mAbs 2013; 5:432-44; Yin et al., Pharmaceutical research 2013; 30:167-78; Li et al., mAbs 2016:0; Li et al., mAbs 2016:0), oxidation (see, for example, Yin et al., Pharmaceutical research 2013; 30:167-78; Li et al., mAbs 2016:0; Li et al., mAbs 2016:0), glycation (see, for example, Goetze et al., Glycobiology 2012; 22:221-34), glycosylation (see, for example, Li et al., mAbs 2016:0; Li et al., mAbs 2016:0; Goetze et al., Glycobiology 2011; 21:949-59; Alessandri et al., mAbs 2012; 4:509-20), disulfides (see, for example, Li Y et al., mAbs 2016:0; Liu et al., The Journal of biological chemistry 2008; 283:29266-72), N-terminal pyroglutamate (see, for example, Yin et al., Pharmaceutical research 2013; 30:167-78; Li et al., mAbs 2016:0; Li et al., mAbs 2016:0; Liu et al., The Journal of biological chemistry 2011; 286:11211-7), and C-terminal lysine removal (see, for example, Li et al., mAbs 2016:0; Cai et al., Biotechnology and bioengineering 2011; 108:404-12) have been investigated in animal or human samples. Accordingly, additional methods of monitoring mAb preparations are needed.

### SUMMARY OF THE INVENTION

A method has been developed for analysis of protein variants, for example size and charge variants of a therapeutic antibody. The method comprises the use of native microfluidic capillary electrophoresis connected in line with mass spectrometry to separate, identify and/or quantify polypeptides in a sample. The method may be used, for example, to characterize post-translational modifications (PTMs) of an antibody, to detect monospecific mAb impurities (referred as either HC/HC or HC*/HC*) in a bispecific antibody (bsAb) sample, and to characterize alternative antibody formats such as “N−1,” which is a one arm antibody, and “N+1” antibodies, where an additional Fab arm was connected to the heavy chain.

In one aspect, the present invention provides a method for detecting and/or discriminating between post-translational modification variants of an antibody of interest in a sample, in which the method includes: contacting a sample comprising one or more antibodies of interest with a protease to digest the sample into antibody fragments; separating antibody fragments by molecular weight and/or charge in one or more capillaries using capillary electrophoresis; eluting separated antibody fragments from the one or more capillaries; and determining the mass of the eluted antibody fragments by mass spec analysis, thereby detecting and/or discriminating between post-translational modification variants of the antibody of interest.

In various embodiments of the method, the post-translational modification comprises one or more of deamidation, oxidation, glycation, disulfide formation, N-terminal pyroglutamate formation, C-terminal lysine removal, high mannose glycosylation, and O-glycosylation.

In various embodiments of the method, the protease comprises IdeS.

In various embodiments of the method, the antibody fragments comprise one or more of an F(ab′)₂ or Fc antibody subunit.

In various embodiments of the method, the antibody of interest is a mAb.

In various embodiments of the method, the antibody fragments are separated by charge and the method is a method of detecting and/or discriminating between charge variants of the antibody of interest.

In various embodiments of the method, the antibody fragments are separated by molecular weight and the method is a method of detecting and/or discriminating between size variants of the antibody of interest.

In some embodiments, the method further includes determining a relative or absolute amount of the post-translational modification variants of an antibody of interest in a sample.

In various embodiments of the method, the antibody of interest comprises a bispecific antibody.

In various embodiments of the method, the sample includes an internal standard.

In various embodiments of the method, the one or more capillaries comprise a separation matrix.

In various embodiments of the method, the separation matrix comprises a sieving matrix configured to separate proteins by molecular weight.

In various embodiments of the method, eluting separated antibody fragments from the one or more capillaries further comprises separating the antibody fragments into one or more fractions.

In some embodiments, the method further includes identifying the antibody fragments.

In some embodiments, the method further includes identifying the post-translational modification present on the antibody fragments.

In various embodiments of the method, the monoclonal antibody of interest is of isotype IgG1, IgG2, IgG3, IgG4, or mixed isotype.

In some embodiments, the method further includes post-translational modification profiling of the antibody of interest.

In some embodiments, the method further includes post-translational modification mapping of post-translational modification hotspots by reduced peptide mapping LC-MS/MS analysis.

In various embodiments of the method, the sample comprises a mixture of antibodies of interest.

In various embodiments of the method, the monoclonal antibody of interest is an antibody drug conjugate.

This disclosure provides a further method for detecting and/or discriminating between post-translational modification variants of an antibody of interest in a sample. In some exemplary embodiments, the method comprises (a) contacting a sample comprising one or more antibodies of interest with a protease to digest the sample into antibody fragments; (b) separating antibody fragments by molecular weight and/or charge in one or more capillaries using capillary electrophoresis; (c) eluting separated antibody fragments from the one or more capillaries; and (d) determining the mass of the eluted antibody fragments by mass spectrometry analysis, thereby detecting and/or discriminating between post-translational modification variants of the antibody of interest, wherein said one or more antibodies of interest are maintained in native conditions, and wherein said capillary electrophoresis is in an integrated microfluidic platform.

In one aspect, the post-translational modification comprises one or more of deamidation, oxidation, glycation, disulfide formation, N-terminal pyroglutamate formation, C-terminal lysine removal, high mannose glycosylation, and O-glycosylation.

In one aspect, the protease comprises IdeS.

In one aspect, the antibody fragments comprise one or more of an F(ab′)₂ or Fc antibody subunit.

In one aspect, the antibody of interest is a monoclonal antibody.

In one aspect, the antibody fragments are separated by charge and the method is a method of detecting and/or discriminating between charge variants of the antibody of interest.

In one aspect, the antibody fragments are separated by molecular weight and the method is a method of detecting and/or discriminating between size variants of the antibody of interest.

In one aspect, the method further comprises determining a relative or absolute amount of the post-translational modification variants of an antibody of interest in a sample.

In one aspect, the antibody of interest comprises a bispecific antibody.

In one aspect, the sample includes an internal standard.

In one aspect, the one or more capillaries comprise a separation matrix. In a specific aspect, said separating comprises a sieving matrix configured to separate proteins by molecular weight.

In one aspect, eluting separated antibody fragments from said one or more capillaries further comprises separating the antibody fragments into one or more fractions.

In one aspect, the method further comprises identifying the antibody fragments.

In one aspect, the method further comprises identifying the post-translational modification present on the antibody fragments.

In one aspect, the antibody of interest is of isotype IgG1, IgG2, IgG3, IgG4, or mixed isotype.

In one aspect, the method further comprises post-translational modification profiling of the antibody of interest.

In one aspect, the method further comprises post-translational modification mapping of post-translational modification hotspots by reduced peptide mapping LC-MS/MS analysis.

In one aspect, the sample comprises a mixture of antibodies of interest.

In one aspect, the antibody of interest is an antibody drug conjugate. In another aspect, the antibody of interest is a “N−1” antibody or a “N+1” antibody.

In one aspect, an injection volume in the one or more capillaries is between about 1 nL and about 10 nL. In a specific aspect, an injection volume in the one or more capillaries is about 1 nL.

This disclosure provides a further method for detecting and/or discriminating between post-translational modification variants of an antibody of interest in a sample. In some exemplary embodiments, the method comprises (a) contacting a sample comprising one or more antibodies of interest with an IdeS protease to digest the sample into antibody fragments, wherein the protease to sample ratio is about 1.25 units of protease to about 1 μg sample; (b) separating antibody fragments by molecular weight and/or charge in one or more capillaries comprising a sieving matrix using capillary electrophoresis; (c) eluting separated antibody fragments from the one or more capillaries; and (d) determining the mass of the eluted antibody fragments by mass spectrometry analysis, thereby detecting and/or discriminating between post-translational modification variants of the antibody of interest, wherein said antibody of interest is maintained in native conditions, and wherein said capillary electrophoresis is in an integrated microfluidic platform.

This disclosure also provides a method for characterizing a monospecific antibody in a mixture of a bispecific antibody and its monospecific antibody side products. In some exemplary embodiments, the method comprises (a) separating a mixture of a bispecific antibody and its monospecific antibody side products by molecular weight and/or charge in one or more capillaries using capillary electrophoresis; (b) eluting said separated antibody and antibody side products from said one or more capillaries; and (c) determining the mass of said eluted antibody and antibody side products by mass spectrometry, thereby characterizing said monospecific antibody, wherein said monospecific antibody is maintained in native conditions, and wherein said capillary electrophoresis is in an integrated microfluidic platform.

In one aspect, the method further comprises determining a relative or absolute amount of the monospecific antibody in said mixture.

In one aspect, the mixture includes an internal standard.

In one aspect, the one or more capillaries comprise a separation matrix. In a specific aspect, the separation matrix comprises a sieving matrix configured to separate proteins by molecular weight.

In one aspect, the monospecific antibody is of isotype IgG1, IgG2, IgG3, IgG4, or mixed isotype.

In one aspect, the method further comprises characterizing a second monospecific antibody in the mixture.

In one aspect, an injection volume in the one or more capillaries is between about 1 nL and about 10 nL. In a specific aspect, an injection volume in the one or more capillaries is about 1 nL.

This disclosure also provides methods for identifying, quantifying, and/or characterizing at least one charge variant of a protein of interest. In some embodiments, the methods can comprise (a) subjecting a sample including a protein of interest to stress conditions to produce a sample including at least one charge variant of said protein of interest; (b) subjecting said sample including at least one charge variant of said protein of interest to native capillary electrophoresis to produce an eluate; and (c) subjecting said eluate to mass spectrometry analysis to identify, quantify, and/or characterize said at least one charge variant of said protein of interest, wherein said native capillary electrophoresis is performed in an integrated microfluidic device directly coupled to said mass spectrometer.

In one aspect, said protein of interest is selected from a group consisting of an antibody, a monoclonal antibody, a monospecific antibody, a bispecific antibody, a multispecific antibody, an antibody fusion protein, an antibody-drug conjugate, an antibody fragment, an antibody subunit, an antigen-binding protein, and antibody-derived protein, and a therapeutic antibody.

In one aspect, a duration of said stress conditions is from 0 hours to 24 hours, from 0 days to 30 days, from 0 weeks to 8 weeks, from 0 months to 12 months, about 1 hour, about 6 hours, about 12 hours, about 18 hours, about 24 hours, about 1 day, about 2 days, about 3 days, about 4 days, about 5 days, about 6 days, about 7 days, about 14 days, about 15 days, about 21 days, about 28 days, about 1 week, about 2 weeks, about 3 weeks, about 4 weeks, about 5 weeks, about 6 weeks, about 7 weeks, about 8 weeks, about 1 month, about 2 months, about 3 months, about 4 months, about 5 months, about 6 months, about 7 months, about 8 months, about 9 months, about 10 months, about 11 months, or about 12 months.

In one aspect, said stress conditions comprise thermal stress, pH stress, oxidation stress, ultraviolet stress, and combinations thereof. In a specific aspect, said thermal stress comprises subjecting said sample to a temperature from 20° C. to 80° C., from 25° C. to 50° C., about 25° C., about 30° C., about 35° C., about 37° C., about 40° C., about 45° C., about 50° C., about 55° C., about 60° C., about 65° C., about 70° C., about 75° C., or about 80° C. In another specific aspect, said pH stress comprises subjecting said sample to a pH from 4 to 10, about 4, about 5, about 6, about 7, about 8, about 9, or about 10. In an additional specific aspect, said oxidation stress comprises contacting said sample to H₂O₂ at a concentration from 0.5 ppm to 20 ppm, about 0.5 ppm, about 1 ppm, about 2 ppm, about 3 ppm, about 4 ppm, about 5 ppm, about 6 ppm, about 7 ppm, about 8 ppm, about 9 ppm, about 10 ppm, about 15 ppm, or about 20 ppm.

In one aspect, said at least one charge variant comprises an acidic variant and/or a basic variant.

In one aspect, the method further comprises attributing said at least one charge variant to at least one post-translational modification. In a specific aspect, said at least one post-translational modification comprises deamidation, oxidation, glycation, disulfide formation, N-terminal pyroglutamate formation, C-terminal lysine removal, high mannose glycosylation, 0-glycosylation, isomerization, truncation, or combinations thereof.

In one aspect, the method further comprises repeating steps (a)-(c) using at least one additional stress condition or control condition.

In one aspect, an injection volume of said sample for said native capillary electrophoresis is from 0.5 nL to 2 nL, about 0.5 nL, about 1 nL, about 1.5 nL, or about 2 nL.

In one aspect, said integrated microfluidic device comprises an integrated electrospray ionization emitter.

In one aspect, the method further comprises subjecting said sample including said at least one charge variant of said protein of interest to digestion conditions prior to step (b). In a specific aspect, said digestion conditions comprise contacting said sample to at least one digestive enzyme selected from a group consisting of pepsin, trypsin, Tryp-N, chymotrypsin, Lys-N, Lys-C, Asp-N, Arg-C, Glu-C, papain, IdeS, or a variant thereof.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

### BRIEF DESCRIPTION OF THE DRAWINGS

FIG. 1 illustrates an exemplary work flow for the separation and detection of post-translational modified antibody fragments by capillary electrophoresis and mass spectral analysis, according to an exemplary embodiment.

FIG. 2A shows electropherograms evaluating the sensitivity of ZipChip nCE-MS for NISTmAb (IgG1), according to an exemplary embodiment.

FIG. 2B shows electropherograms evaluating the sensitivity of ZipChip nCE-MS for mAb1 (IgG1), according to an exemplary embodiment.

FIG. 2C shows electropherograms evaluating the sensitivity of ZipChip nCE-MS for mAb2 (IgG4), according to an exemplary embodiment.

FIG. 2D shows electropherograms evaluating the sensitivity of ZipChip nCE-MS for bsAb1 (IgG4), according to an exemplary embodiment.

FIG. 3 shows mass spectra at the limit of detection (LOD) for NISTmAb, mAb1, mAb2, and bsAb1, according to an exemplary embodiment.

FIG. 4A shows sensitivity of ZipChip nCE-MS for IgG1, according to an exemplary embodiment.

FIG. 4B shows sensitivity of ZipChip nCE-MS for IgG4, according to an exemplary embodiment.

FIG. 4C shows a carryover test of ZipChip nCE-MS, according to an exemplary embodiment.

FIG. 5A shows a charge variant profile of deglycosylated NISTmAb, including two basic variants and two acidic variants, according to an exemplary embodiment.

FIG. 5B shows a mass spectrum of the acidic 2 variant of NISTmAb, demonstrating Fab glycosylation, according to an exemplary embodiment.

FIG. 5C shows a zoom-in view of a deglycosylated NISTmAb electropherogram, showing three truncated forms, according to an exemplary embodiment.

FIG. 5D shows mass spectra of three NISTmAb truncation forms cleaved at (i) Cys223/Asp224, (ii) Lys225/Thr226, and (iii) His227/Thr228, according to an exemplary embodiment.

FIG. 6 shows the charge variant separation of intact NISTmAb and SEQ ID NO: 1, according to an exemplary embodiment.

FIG. 7A shows an electropherogram of charge variant analysis of antibody F(ab′)2 and Fc subunits for IdeS treated control and stressed NISTmAb, according to an exemplary embodiment.

FIG. 7B shows an electropherogram of charge variant analysis of antibody F(ab′)2 subunits for IdeS treated control and stressed NISTmAb, according to an exemplary embodiment.

FIG. 7C shows an electropherogram of charge variant analysis of antibody Fc subunits for IdeS treated control and stressed NISTmAb, according to an exemplary embodiment.

FIG. 8A shows a separation of three IgG1 mAbs using ZipChip nCE-MS, according to an exemplary embodiment.

FIG. 8B shows a separation of five bispecific IgG4 mAbs using ZipChip nCE-MS, according to an exemplary embodiment.

FIG. 8C shows a separation of ten mAbs using ZipChip nCE-MS, according to an exemplary embodiment.

FIG. 8D shows an identification of co-migrated mAbs using ZipChip nCE-MS, according to an exemplary embodiment.

FIG. 8E shows an identification of co-migrated mAbs using ZipChip nCE-MS, according to an exemplary embodiment.

FIG. 9 shows electropherograms for five lots of formulated antibodies manufactured from two different processes, according to an exemplary embodiment.

FIG. 10 shows mass spectra of main species and basic variant 1 for five lots of mAb3 manufactured with two different processes, according to an exemplary embodiment.

FIG. 11A shows charge variant separation by SCX-UV, according to an exemplary embodiment.

FIG. 11B shows charge variant separation by ZipChip nCE-MS, according to an exemplary embodiment.

FIG. 12A shows charge variant separation by iCIEF, according to an exemplary embodiment.

FIG. 12B shows charge variant separation by ZipChip nCE-MS, according to an exemplary embodiment.

FIG. 13A shows an electropherogram of bsAb1 with 20 ng injection by ZipChip nCE-MS, according to an exemplary embodiment.

FIG. 13B shows a 10× zoom-in of an electropherogram of bsAb1 with 20 ng injection by ZipChip nCE-MS, according to an exemplary embodiment.

FIG. 13C shows HC*/HC* monospecific mAb impurities at spike-in levels of 1:500 and 1:100 in bsAb1 by ZipChip nCE-MS, according to an exemplary embodiment.

FIG. 13D shows a zoom-in view of HC*/HC* monospecific mAb impurities at spike-in levels of 1:500 and 1:100 in bsAb1 by ZipChip nCE-MS, according to an exemplary embodiment.

FIG. 14 shows mass spectra for HC/HC in a bsAb1 sample, HC/HC standard, and half mAb by ZipChip nCE-MS, according to an exemplary embodiment.

FIG. 15 shows mass spectra of HC*/HC* at 0.2% and 1% using ZipChip nCE-MS, according to an exemplary embodiment.

FIG. 16A shows an electropherogram of an “N−1” format antibody using ZipChip nCE-MS, according to exemplary embodiment.

FIG. 16B shows an electropherogram of an “N+1” format antibody using ZipChip nCE-MS, according to an exemplary embodiment.

FIG. 17 shows identification of O-glycosylation (Xyl+Gal+Gal+GlcA) in acidic variant 1 of mAb7 using ZipChip nCE-MS, according to an exemplary embodiment.

FIG. 18A shows electropherograms of NISTmAb charge variants after thermal stress, according to an exemplary embodiment.

FIG. 18B shows clipping sites of NISTmAb observed under thermal stress conditions, according to an exemplary embodiment.

FIG. 18C shows an identification of Fab arms of NISTmAb charge variants under thermal stress conditions, according to an exemplary embodiment.

FIG. 18D shows an identification of mAb-Fab arms of NISTmAb charge variants under thermal stress conditions, according to an exemplary embodiment.

FIG. 19 shows deconvoluted mass spectra of NISTmAb fragments under thermal stress conditions, according to an exemplary embodiment.

FIG. 20A shows electropherograms of NISTmAb F(ab′)2 fragment charge variants after thermal stress, according to an exemplary embodiment.

FIG. 20B shows electropherograms of NISTmAb Fc fragment charge variants after thermal stress, according to an exemplary embodiment.

FIG. 21A shows electropherograms of mAb8 charge variants after thermal stress, according to an exemplary embodiment.

FIG. 21B shows a zoomed-in view of electropherograms of mAb8 charge variants after thermal stress, according to an exemplary embodiment.

FIG. 21C shows PTM identifications proposed for each mAb8 charge variant after thermal stress, according to an exemplary embodiment.

FIG. 22A shows deconvoluted mass spectra of main, b0, and B0 charge variants of mAb8 after 3 months of thermal stress, according to an exemplary embodiment.

FIG. 22B shows deconvoluted mass spectra of main, b0, and B0 charge variants of mAb8 after 3 weeks of thermal stress, according to an exemplary embodiment.

FIG. 22C shows deconvoluted mass spectra of main, b0, and B0 charge variants of mAb8 after 5 weeks of thermal stress, according to an exemplary embodiment.

FIG. 23A shows electropherograms of bsAb4 charge variants after oxidative stress, according to an exemplary embodiment.

FIG. 23B shows PTM identifications proposed for each bsAb4 charge variant after oxidative stress, according to an exemplary embodiment.

FIG. 23C shows mass spectra of bsAb4 basic variants after oxidative stress, according to an exemplary embodiment.

FIG. 24A shows electropherograms of bsAb4 charge variants after pH stress, according to an exemplary embodiment.

FIG. 24B shows mass spectra of bsAb4 charge variants after pH stress, according to an exemplary embodiment.

FIG. 24C shows mass spectra of bsAb4 charge variants after pH stress, according to an exemplary embodiment.

FIG. 24D shows PTM identifications proposed for each bsAb4 charge variant after pH stress, according to an exemplary embodiment.

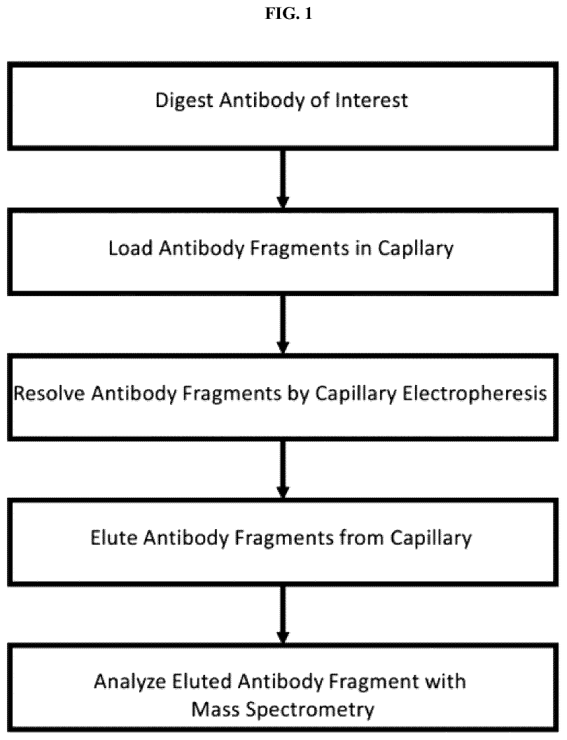

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

### Abbreviations Used Herein

mAb: Monoclonal antibody
biAb/bsAb: Bispecific antibody
CQA: Critical quality attributes
CE: Capillary Electrophoresis
PTM: Post-Translational Modification
IEC: Ion Exchange Chromatography
UV: Ultra Violet
QC: Quality Control
MS: Mass Spectrometry
ADC: Antibody Drug Conjugate

The term “antibody”, as used herein, is intended to refer to immunoglobulin molecules included of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is included of a heavy chain variable region (“HCVR” or “V_{H}”) and a heavy chain constant region (included of domains C_{H}1, C_{H}2 and C_{H}3). In various embodiments, the heavy chain may be an IgG isotype. In some cases, the heavy chain is selected from IgG1, IgG2, IgG3 or IgG4. In some embodiments, the heavy chain is of isotype IgG1 or IgG4, optionally including a chimeric hinge region of isotype IgG1/IgG2 or IgG4/IgG2. Each light chain is included of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The term “antibody” includes reference to both glycosylated and non-glycosylated immunoglobulins of any isotype or subclass. The term “antibody” includes antibody molecules prepared, expressed, created or isolated by recombinant means, such as antibodies isolated from a host cell transfected to express the antibody. For a review on antibody structure, see Lefranc et al., IMGT unique numbering for immunoglobulin and T cell receptor variable domains and Ig superfamily V-like domains, 27(1) Dev. Comp. Immunol. 55-77 (2003); and M. Potter, Structural correlates of immunoglobulin diversity, 2(1) Surv. Immunol. Res. 27-42 (1983).

The term antibody also encompasses a “bispecific antibody”, which includes a heterotetrameric immunoglobulin that can bind to more than one different epitope. One half of the bispecific antibody, which includes a single heavy chain and a single light chain and six CDRs, binds to one antigen or epitope, and the other half of the antibody binds to a different antigen or epitope. In some cases, the bispecific antibody can bind the same antigen, but at different epitopes or non-overlapping epitopes. In some cases, both halves of the bispecific antibody have identical light chains while retaining dual specificity. Bispecific antibodies are described generally in U.S. Patent App. Pub. No. 2010/0331527 (Dec. 30, 2010).

Co-expression of two unique heavy chains (HC and HC*) and one common light chain will minimize a number of side products to two monospecific mAb impurities, which may need to be subsequently detected and removed during purification. The two impurities may be referred to as HC/HC and HC*/HC* respectively. In some exemplary embodiments, the CH3 domain of one heavy chain contains amino acid substitutions that prevent binding to protein A, for example H95R and Y96F substitutions. In some exemplary embodiments, the method of the present invention can be used to detect, identify, quantify, and/or remove monospecific mAb impurities in a sample comprising a bispecific antibody.

The term antibody also encompasses alternative antibody formats including, for example, “N−1”, which is a one arm antibody, and “N+1”, where an additional Fab arm is connected using, for example, a G4S linker. The method of the present invention can also be used in the analysis of, for example, antibody subunits, antibody fusion proteins, single chain variable fragments (scFvs), diabodies, triabodies, and other antigen-binding proteins or any protein of interest.

The term “antigen-binding portion” of an antibody (or “antibody fragment”), refers to one or more fragments of an antibody that retain the ability to specifically bind to an antigen. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et at. (1993) 90 PNAS U.S.A. 6444-6448; and Poljak et at. (1994) 2 Structure 1121-1123).

Moreover, antibodies and antigen-binding fragments thereof can be obtained using standard recombinant DNA techniques commonly known in the art (see Sambrook et al., 1989).

The term “human antibody”, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “ADC” or “antibody-drug conjugate” refers to an antibody or antigen-binding fragment thereof conjugated to a therapeutic moiety such as a cytotoxic agent, a chemotherapeutic drug, immunosuppressant or a radioisotope. Cytotoxic agents include any agent that is detrimental to the growth, viability or propagation of cells. Examples of suitable cytotoxic agents and chemotherapeutic agents for forming ADCs are known in the art.

The term “sample,” as used herein, refers to a mixture of molecules that includes at least one polypeptide of interest, such as a monoclonal antibody or a bispecific antibody or fragment thereof, that is subjected to manipulation in accordance with the methods of the invention, including, for example, separating, analyzing, extracting, concentrating or profiling.

The terms “analysis” or “analyzing,” as used herein, are used interchangeably and refer to any of the various methods of separating, detecting, isolating, purifying, solubilizing, detecting and/or characterizing molecules of interest (e.g., polypeptides, such as antibodies) and contaminants in antibody preparations. Examples include, but are not limited to, electrophoresis, mass spectrometry, e.g. tandem mass spectrometry, ultraviolet detection, and combinations thereof.

“Chromatography,” as used herein, refers to the process of separating a mixture, for example a mixture containing peptides, proteins, polypeptides and/or antibodies, such as monoclonal antibodies. It involves passing a mixture through a stationary phase, which separates molecules of interest from other molecules in the mixture and allows one or more molecules of interest to be isolated.

The term “isolated,” as used herein, refers to a biological component (such as an antibody, for example a monoclonal antibody) that has been substantially separated, produced apart from, or purified away from other biological components in the cell of the organism in which the component naturally occurs or is transgenically expressed, that is, other chromosomal and extrachromosomal DNA and RNA, proteins, lipids, and metabolites. Nucleic acids, peptides, proteins, lipids and metabolites which have been “isolated” thus include nucleic acids, peptides, proteins, lipids, and metabolites purified by standard or non-standard purification methods. The term also embraces nucleic acids, peptides, proteins, lipids, and metabolites prepared by recombinant expression in a host cell as well as chemically synthesized peptides, lipids, metabolites, and nucleic acids.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan W Trp; and Tyrosine Y Tyr. In one embodiment, a peptide is an antibody or fragment or part thereof, for example, any of the fragments or antibody chains listed above. In some embodiments, the peptide may be post-translationally modified. As used herein, the terms “protein of interest” and/or “target protein of interest” refer to any protein to be separated and/or detected with the methods provided herein. Suitable proteins of interest include antibodies, for example monoclonal antibodies, and fragments thereof.

“Detect” and “detection” have their standard meaning, and are intended to encompass detection including the presence or absence, measurement, and/or characterization of a protein of interest, such as a mAb or fragment thereof.

As used herein, the terms “standard” and/or “internal standard” refer to a well-characterized substance of known amount and/or identity (e.g., known molecular weight, electrophoretic mobility profile) that can be added to a sample and both the standard and the molecules in the sample, on the basis of molecular weight or isoelectric point by electrophoresis). A comparison of the standard then provides a quantitative or semi-quantitative measure of the amount of analyte, such as mAb or fragments thereof present in the sample.

“Contacting,” as used herein, includes bringing together at least two substances in solution or solid phase, for example contacting a sample with an enzyme, such as a protease.

The term “corresponding” is a relative term indicating similarity in position, purpose or structure, and may include peptides of identical structure but for the presence or absence of a post-translational modification. In some embodiments, mass spectral signals in a mass spectrum that are due to corresponding peptides of identical structure but for the presence or absence of a post-translational modification are “corresponding” mass spectral signals. A mass spectral signal due to a particular peptide is also referred to as a signal corresponding to the peptide. In certain embodiments, a particular peptide sequence or set of amino acids can be assigned to a corresponding peptide mass.

The terms “fragment peptide” or “peptide fragment,” as used herein, refer to a peptide that is derived from the full-length polypeptide, such as a protein and/or monoclonal antibody, through processes including fragmentation, enzymatic proteolysis, or chemical hydrolysis. Such proteolytic peptides include peptides produced by treatment of a protein with one or more proteases, such as IdeS protease. A fragment peptide, or peptide fragment, can be a digested peptide. A fragment of an antibody produced by treatment with a protease such as IdeS or papain may be referred to as a subunit, and analysis of an antibody subunit may be referred to as subunit analysis.

“Mass spectrometry” refers to a method in which a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), orbitrap mass analyzer, Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography.

Tandem mass spectrometry or MS/MS is a technique to break down selected ions (precursor ions) into fragments (product ions). The fragments then reveal aspects of the chemical structure of the precursor ion. In tandem mass spectrometry, once samples are ionized (for example by ESI, MALDI, EI, etc.) to generate a mixture of ions, precursor ions, for example peptides from a digest of a specific mass-to-charge ratio (m/z) are selected (MS1) and then fragmented (MS2) to generate product ions for detection. Typical tandem MS instruments include QqQ, QTOF, and hybrid ion trap/FTMS, etc. One example of an application of tandem mass spectrometry is protein identification. The first mass analyzer isolates ions of a particular m/z value that represent a single species of peptide among many introduced into and then emerging from the ion source. Those ions are then accelerated into a collision cell containing an inert gas such as argon to induce ion fragmentation. This process is designated collisionally induced dissociation (CID) or collisionally activated dissociation (CAD). The m/z values of fragment ions are then measured in a 2^{nd} mass analyzer to obtain amino acid sequence information.

References to a mass of an amino acid mean the monoisotopic mass or average mass of an amino acid at a given isotopic abundance, such as a natural abundance. In some examples, the mass of an amino acid can be skewed, for example, by labeling an amino acid with an isotope. Some degree of variability around the average mass of an amino acid is expected for individual single amino acids based on the exact isotopic composition of the amino acid. The masses, including monoisotopic and average masses for amino acids are easily obtainable by one of ordinary skill the art.

Similarly, references to a mass of a peptide means the monoisotopic mass or average mass of a peptide at a given isotopic abundance, such as a natural abundance. In some examples, the mass of a peptide can be skewed, for example, by labeling one or more amino acids in the peptide with an isotope. Some degree of variability around the average mass of a peptide is expected for individual single peptides based on the exact isotopic composition of the peptide. The mass of a particular peptide can be determined by one of ordinary skill the art.

In some exemplary embodiments, CE, MS, and CE-MS can be performed under native conditions (nCE-MS). As used herein, the term “native conditions” can include performing capillary electrophoresis and/or mass spectrometry under conditions that preserve non-covalent interactions in an analyte, in contrast to, for example, reducing and/or denaturing conditions. Native capillary electrophoresis and/or mass spectrometry is an approach to study intact biomolecular structure in the native or near-native state. The term “native” refers to the biological status of the analyte in solution prior to subjecting to separation and/or ionization. Several parameters, such as pH and ionic strength, of the solution containing the biological analytes can be controlled to maintain the native folded state of the biological analytes in solution.

In some aspects, the sample including the protein of interest can be prepared prior to or following enrichment steps, separation steps, and/or analysis steps. Preparation steps can include alkylation, reduction, denaturation, digestion, desalting, deglycosylation, and other steps.

As used herein, “protein denaturing” refers to a process in which the three-dimensional shape of a molecule is changed from its native state. Protein denaturation can be carried out using a protein denaturing agent. Non-limiting examples of a protein denaturing agent include heat, high or low pH, reducing agents like DTT or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, IdeS or a variant thereof is used to cleave an antibody below the hinge region, producing an Fc fragment and a Fab2 fragment. Digestion of an analyte may be advantageous because size reduction may increase the sensitivity and specificity of characterization and detection of the analyte using CE-MS or LC-MS. When used for this purpose, digestion that separates out an Fc fragment and keeps a Fab2 fragment for analysis may be preferred. This is because variable regions of interest, such as the complementarity-determining region (CDR) of an antibody, are contained in the Fab2 fragment, while the Fc fragment may be relatively uniform between antibodies and thus provide less relevant information. Alternatively, or additionally, digestion that separates out a Fab2 fragment and keeps an Fc fragment for analysis may be preferred, because the Fc fragment contains an N-glycosylation site of interest.

IdeS digestion has a high efficiency, allowing for high recovery of an analyte. IdeS or variants thereof are commercially available and may be marketed as, for example, FabRICATOR® or FabRICATOR Z®.

General Description

Therapeutic antibodies are a major class of biopharmaceuticals that have been developed as treatments for a variety of therapeutic areas including infectious diseases (Sparrow et al., 2017, Bull. World Health Organ., 95(3):235-237), inflammation and immunology (Chan et al., 2010, Nat. Rev. Immunol., 10(50):301-316), oncology (Scott et al., 2012, Nat. Rev. Cancer., 12(4):278-287), hematology (Cuesta-Mateos et al., 2017, Front. Immunol., 8:1936), ophthalmology (Rodrigues et al., 2009, Prog. Retin. Eye Res.), 28(2):117-144, and rare diseases (Tambuyzer et al., 2020, Nat. Rev. Drug Discov., 19(2):93-111). The advantages of therapeutic antibodies include the high specificity and binding affinity to their molecular targets, and generally low immunotoxicity and mild adverse effects (Rodrigues et al.; Lu et al., 2020, J. Biomed. Sci., 27(1):1). Monoclonal antibodies (mAbs) including immunoglobulin G (IgG) subclasses, such as IgG1 and IgG4, have attracted the most attention, because of their simpler structures and longer half-lives than those of other subclasses. The structure of these antibodies consists of two identical heavy chains and light chains, with intrachain and interchain disulfide bond linkage (Chiu et al., 2019, Antibodies (Basel), 8:55). Additionally, bispecific antibodies (bsAbs), in which the two heavy chains and light chains may be different, and other IgG-based molecules in alternative formats, such as those with addition and removal of the antigen-binding fragment (Fab) arm, have also been created to achieve advantageous properties such as the ability to interact with multiple therapeutic targets (Labrijn et al., 2019, Nat. Rev. Drug Discov., 18(8):585-608; Seung et al., 2022, Nature, 603(7900):328-334; Spiess et al., 2015, Mol. Immunol., 67(95-106)). In cancer treatment, bsAbs can achieve high target specificity through one paratope and recruitment of tumor killing agents through the other paratope (Runcie et al., 2018, Mol. Med., 24(1):50).

Over the course of manufacturing, processing, storage, and clinical administration, therapeutic antibodies are subjected to various types of stress conditions and factors. These factors include storage and use temperature, transit-based agitation, oxidation from the cleaning agents used during the manufacturing process, and ambient light exposure during storage and administration (Das et al., 2020, J Pharm Sci, 109(1):116-133). Forced degradation and stability studies are conducted to evaluate the risk of degradation and the effects of these commonly encountered stresses, to understand and consequently protect against these stressors (Hawe et al., 2012, J Pharm Sci, 101(3):895-913; Nowak et al., 2017, MAbs, 9(8):1217-1230; Torrente-Lopez et al., 2022, Pharmaceutics, 14(4)). The test results are often used to provide information during quality attribute risk assessment, thus aiding in the identification of preliminary critical quality attributes to be monitored during product development and release control (Kuzman et al., 2021, Sci Rep, 11(1):20534).

Characterization of antibody variants is important in order to identify their potential impact on safety, potency, and stability of a potential therapeutic antibody. For example, to be considered for approval by regulatory agencies, extensive characterization of the molecule must be performed. In drug products comprising mixtures of antibodies, characterization of the absolute or relative amounts of each antibody must be determined. Because aggregates and fragments may potentially affect immunogenicity and potency, their levels are typically monitored during lot release, stability, and characterization. Furthermore, primary degradation pathways for the molecule and product related impurities and variants are determined.

Characterization of these therapeutic antibodies and their derivatives has become more challenging, owing to the increasing complexity of these biomolecules and their heterogeneity. For example, post-translational modifications (PTMs) commonly occur on therapeutic antibodies in bioreactors during production (Wurm, 2004, Nat. Biotechnol., 22(11):1393-1398). These modifications include glycosylation, asparagine deamidation, aspartic acid isomerization and cyclization, methionine and tryptophan oxidation, and lysine glycation, among others (Liu et al., 2008, J. Pharm. Sci., 97(7):2426-2447). As amino acid residues are modified, the electrostatic environment surrounding the amino acids changes, thereby resulting in a net charge difference in the molecule and the formation of charge variants (Du et al., 2012, MAbs, 4(5):578-585). For instance, lysine glycation adds a neutrally charged monosaccharide to a positively charged and basic lysine residue, thus resulting in an acidic variant. Methionine oxidation changes a methionine residue to a methionine sulfone, thus resulting in a basic variant. Moreover, cellular processing also causes the formation of charge variants, including incomplete cleavage of C-terminal lysine, and incomplete cyclization of the N-terminal glutamine residue. Mass spectrometry (MS) has been extensively used to study PTMs at the peptide, subunit, and intact levels (Zhang et al., 2014, FEBS Lett., 588(2):308-317). Peptide mapping, wherein therapeutic antibodies are digested with enzymes, can be used to monitor multiple PTMs simultaneously and have been widely adopted (Rogstad et al., 2017, J. Am. Soc. Mass Spectrom., 28(5):786-794). Additionally, intact mass analysis of therapeutic antibodies at both the subunit and intact levels has also been performed (Jin et al., 2019, MAbs, 11(1): 106-115).

Charge variant analysis is critical for understanding the structure of biotherapeutics during drug development and product release (Du et al.). Charge variant analysis provides information on the electrostatic environment surrounding amino acids, according to the net charge difference between unmodified and modified forms of therapeutic antibodies. Degradation of therapeutic antibodies can manifest as changes in amino acid levels in the form of post-translational modifications (PTMs), and sequence truncations, thus forming charge variants (Du et al.; Gupta et al., 2022, J Pharm Sci, 111(4):903-918). In terms of PTMs, acidic variants can arise from PTMs such as deamidation, wherein a neutrally charged glutamine is changed to a negatively charged glutamate acid, and glycation, wherein a positively charged lysine is modified with a monosaccharide, thereby generating a neutrally charged moiety. Basic variants can arise from unprocessed C-terminal lysines carrying an additional positive charge with respect to the processed counterpart. Charge variants can also come from sequence truncations, which can be either acidic or basic (Du et al.).

Charge variant profiles can be characterized by several analytical techniques, including imaged capillary isoelectric focusing (iCIEF) and cation exchange chromatography (CEX), to enable relative quantification of charge variants. The iCIEF technique separates charge species according to the isoelectric point (pI) (Salas-Solano et al., 2011, Chromatographia, 73(11):1137-1144). CEX is a chromatographic technique that separates species according to surface charge, and strong cation exchange (SCX) is a common method used for therapeutic antibody analysis (Fekete et al., 2015, J. Pharm. Biomed. Anal., 113:43-55). For iCIEF, electropherograms are generated according to optical detection, and thus the identity of the separated charge variants is not determined. Although several iCIEF-MS platforms have been demonstrated to provide pI based separation and charge variant identification (Mack et al., 2019, Electrophoresis, 40(23-24):3084-3091; He et al., 2022, Electrophoresis), these platforms remain under development and are not widely adopted. Recent advances have enabled direct coupling of CEX to MS through use of MS-compatible salt; however, higher resolution separation is normally achieved with MS-incompatible buffer rather than MS-compatible buffer (Yan et al., 2018, Anal. Chem., 90(21):13013-13020; Ma et al., 2020, MAbs, 12(1):1763762).

Mass spectrometry has become a primary tool for studying therapeutic antibody PTMs at the molecular level (Rogstad et al.). Mass spectrometry-based analyses—including peptide mapping analysis, in which therapeutic antibodies are digested into peptides that are evaluated at a local level (Millan-Martin et al., 2023, Nat Protoc, 18(4):1056-1089), and intact mass analysis, wherein therapeutic antibodies are analyzed at a global level—are routinely used to study therapeutic antibodies (Haberger et al., 2021, J. Am. Soc. Mass Spectrom., 32(8):2062-2071). ZipChip is an integrated microfluidic capillary electrophoresis (CE) system that can be directly coupled with the MS interface, and has been used to analyze small molecules in media, monosaccharides and oligosaccharides (Ribeiro da Silva et al., 2021, J. Chromatogr. A, 1651:462336; Khatri et al., 2017, Anal. Chem., 89(12):6645-6655); peptides for antibody protein sequence confirmation and proteomic analysis (Khatri et al.; Dykstra et al., 2021, J. Am. Soc. Mass Spectrom., 32(8):1952-1963; Cao et al., 2021, J. Pharm. Biomed. Anal., 204:114251); and intact proteins including fusion proteins (Deyanova et al. 2021, Electrophoresis, 42(4):460-464), mAbs (Cao et al.; Sun et al., 2021, Anal. Biochem., 625:114214; Redman et al., 2015, Anal. Chem., 87(4):2264-2272), and antibody-drug conjugates (Redman et al., 2016, Anal. Chem., 88(4):2220-2226). ZipChip is a microfluidic device integrating CE with ESI, enabling preparation, separation, and ESI of samples for MS analysis (Wu et al., 2023, J Pharm Biomed Anal, 223:115147).

Due to the low flow nature of CE and high ionization efficiency of nano-electrospray, this integrated system is able to provide high resolution separation and high sensitivity detection. With the native CE-MS (nCE-MS) method, charge variants are separated on the basis of both protein charge and size under native conditions, on a microchip (Redman et al., 2015). During separation, the charge variants with more positive charges migrate faster and elute earlier in the native BGE (pH<pI of antibody), whereas variants with fewer positive charges migrate slowly in the separation channel under the electric field. Therefore, in a typical antibody charge separation profile, basic variants elute first, followed by the main species and acid variants. Additionally, the ZipChip interface is compatible with common formulation buffer components containing anionic or nonionic detergents for easy sample preparation. Charge variant analysis has been performed with the ZipChip nCE-MS method for monoclonal antibodies (mAbs) including immunoglobin 1 (IgG1), IgG4 and bispecific antibodies (bsAbs) (Wu et al.; Cao et al.; Sun et al.). In addition to identifying PTMs associated with charge variants, ZipChip nCE-MS analysis can uncover truncated forms of therapeutic antibodies in the analysis of charge variants (Wu et al.).

Disclosed herein is a highly sensitive, high-resolution microfluidic nCE-MS method for detecting and/or discriminating between variants of an antibody of interest, such as a therapeutic antibody, in a sample by a physical parameter, such as mass and/or charge. The method may be used, for example, for antibody charge variant and impurity analysis, and for the quick identification of antibody charge variants for forced degradation and long-term stability studies. The sensitivity was first evaluated with three types of therapeutic antibodies. Additionally, antibody samples from different manufacturing processes and lots, as well as the antibody cocktail, were analyzed with the nCE-MS method. Comparative analysis of charge variant profiles was performed for the nCE-MS, SCX and iCIEF methods. The high sensitivity of the nCE-MS method enabled detection of fragments with very low abundance in the antibody sample and homospecific mAb impurities in the bsAb sample. New antibody modalities were also evaluated with the nCE-MS method.

Additionally, the Examples set forth below describe the development of a method including ZipChip nCE-MS analysis to examine therapeutic antibodies under various stress conditions, including thermal stress, oxidative stress, and pH stress. Changes in charge heterogeneity were further investigated, and the underlying changes were elucidated at the molecular level, including by comparison to reduced peptide mapping analysis. It was discovered that the charge heterogeneity of NISTmAb under thermal stress arose from truncations near the hinge region and deamidation. Moreover, subjecting mAb8 to thermal stress resulted in the formation of charge variants primarily through PTMs including pyroglutamination, oxidation, and deamidation. Under oxidative stress, the primary change in the charge variant observed in the case of bsAb4 was caused by oxidation. Under pH stress, various PTMs, including pyroglutamination, oxidation, and deamidation, contributed to the changes in the charge variant profile of bsAb4.

The disclosed methods can be used in QC evaluation of antibody preparations. In embodiments of the method, a sample that includes an antibody of interest is resolved or separated by using capillary electrophoresis, for example on one or more capillaries of a CE-system. In certain embodiments, the sample is resolved or separated by molecular weight and charge. For example, using separation by mass and charge, or m/z ratio, fragments with the same mass but different charges can be resolved. Similarly, using separation by mass and charge, or m/z ratio, fragments with the same charge but different masses can be resolved. In embodiments, the method includes liberating fragments of an antibody of interest, such as a monoclonal antibody (mAb), for example by contacting the sample comprising one or more antibodies of interest with a protease to digest the sample. In an embodiment, the protease is IdeS protease. Once digestion, either partial or full, is conducted, antibody fragments can be separated by molecular weight and/or charge in one or more capillaries using capillary electrophoresis. The separated antibody fragments can be eluted from the one or more capillaries and the mass of the eluted antibody fragments determined by mass spec analysis to detect and/or discriminate between post-translational modification variants of the antibody of interest, for example by detection and/or determination of the PTM profile of the fragments of the antibody of interest. In certain embodiments, the antibody fragments include one or more of an F(ab′)₂ or Fc antibody subunit, for example as digested from the intact antibody using a protease, such as the IdeS protease. In certain embodiments, the antibody of interest is a monoclonal antibody, such as a currently used therapeutic antibody or one undergoing evaluation, including novel monoclonal antibodies. In certain embodiments, the monoclonal antibody of interest is part of an antibody drug conjugate (ADC). In certain embodiments, the antibody fragments are separated by charge and the method is a method of detecting and/or discriminating between charge variants of the antibody of interest. In certain embodiments, the antibody fragments are separated by molecular weight and the method is a method of detecting and/or discriminating between size variants of the antibody of interest. In certain embodiments, the antibody fragments are separated by charge and molecular weight and the method is a method of detecting and/or discriminating between charge and molecular weight variants of the antibody of interest. In certain embodiments, the method includes determining a relative or absolute amount of the post-translational modification variants of an antibody of interest in a sample, for example from the antibody fragments.

As noted above, separation of the antibody fragments by mass and charge has the benefit of being able to determine the homogeneity of the antibody fragments, for example, changes in surface charge of the antibody that may not be easily seen in separation by just molecular weight. This separation allows for the determination of the type and level of post-translational modification on the fragments in the sample. The presence of post-translational modifications (PTMs) on a monoclonal antibody (mAb) induces charge heterogeneity (see Table 1) and potentially affects drug stability and biological activity. Therefore, monitoring the PTMs and associated charge variant profiles of mAbs during drug development is important. Disclosed herein is the development of a high-throughput and highly sensitive native microfluidic CE-MS method for the quick identification of mAb charge variants and its application to forced degradation and long-term stability studies. Relative to ion exchange chromatography (IEX) based approaches, high resolution with comparable charge variant profiles can be obtained using the native microfluidic CE-MS method as disclosed herein.

TABLE 1
Source of antibody charge heterogeneity

| Major PTMs/Degradation | | Species |
|---|---|---|
| Pathway | Effect | Formed |
| Sialylation | COOH addition | Acidic |
| Deamidation | COOH formation | Acidic |
| C-terminal lysine | Loss of NH2 | Acidic |
| cleavage | | |
| Adduct formation | COOH formation or | Acidic |
| | loss of NH2 | |
| Succinimide formation | Loss of COOH | Basic |
| Methionine, cysteine, | Conformational change | Basic |
| lysine, histidine, | | |
| tryptophan oxidation | | |
| Disulfide-mediated | Conformational change | Basic |
| Asialylation (terminal | Loss of COOH | Basic |
| Galactose) | | |
| C-terminal lysine and | NH2 formation or | Basic |
| glycine amidation | loss of COOH | |

In certain embodiments, the post-translational modification is one or more of deamidation, oxidation, glycation, disulfide formation, N-terminal pyroglutamate formation, C-terminal lysine removal, high mannose glycosylation, and O-glycosylation.

In certain embodiments, the sample is resolved or separated within a single capillary. In certain embodiments, the sample is resolved or separated within multiple capillaries, for example in parallel. By way of example with respect to separation by molecular weight, the smaller the fragment of an antibody, the further within a capillary it would be expected to travel over a given period of time. In addition, one would expect differences in the charge of antibody fragments to be subjected to different travel times depending on the charge.

In embodiments, the sample may contain multiple, such as at least 2, at least 3, at least 4, at least 5 or more sets of antibody fragments from multiple antibodies of interest. In some embodiments, the method further includes determining a relative or absolute amount of the variants of the antibody fragments in a sample, for example by measurement of peak height or area, which corresponds to the amount of antibody fragment in the sample. In some embodiments, the antibody of interest comprises a bispecific monoclonal antibody. In some embodiments, the sample includes one or more internal standards, for example a ladder of molecular weight standards, a ladder of isoelectric point standards, or even a standard used as a baseline or benchmark for determining the amount of an antibody fragments of interest in the sample.

The ability to discriminate between mAbs in a mAb cocktail of multiple mAbs is becoming increasingly important as these multiple component therapies demonstrate increased efficacy in disease treatment. Thus, improved methods of monitoring how the individual mAbs behave in these systems will become increasingly important in the assessment of the compatibility and stability of these multi-mAb therapies. To meet this growing need this disclosure provides a method for detecting and/or discriminating between monoclonal antibodies in a mixture of two of more monoclonal antibodies in a sample.

In embodiments, the method includes separating protein components of a sample with two or more mAbs of interest, such as 2, 3, 4, 5, 6, 7, 8, 9 10 or even more, mAbs of interest, by charge in one or more capillaries using capillary electrophoresis

In some embodiments, a charge based profile or fingerprint of the antibody of interest can be created, for example of the antibody of interest alone for comparison with a charge based profile or fingerprint of the antibody in the mixture, for example a charge based profile or fingerprint corresponding to the post-translational modification. This comparison can then be used to determine if the antibody of interest changes in the mixture. This profile or fingerprint comparison can be done for any or all of the antibodies of interest in the mixture.

Samples for use in the disclosed methods can be heterogeneous, containing a variety of components, i.e. different proteins. Alternatively, the sample can be homogenous, containing one component or essentially one component of multiple charge or molecular weight species. Pre-analysis processing may be performed on the sample prior to detecting the antibody of interest, such as a mAb or multiple mAbs. For example, the sample can be subjected to a lysing step, denaturation step, heating step, purification step, precipitation step, immunoprecipitation step, column chromatography step, centrifugation, etc. In some embodiments, the separation of the sample and immobilization may be performed on native substrates. In other embodiments, the sample may be subjected to denaturation, for example, heat and/or contact with a denaturizing agent. Denaturing agents are known in the art. In some embodiments, the sample may be subjected to non-reducing conditions. In some embodiments, the sample may be subjected to reducing conditions, for example, by contacting the sample with one or more reducing agents. Reducing agents are knowns in the art.

In some aspects, the methods and systems disclosed herein may be used for analysis of intact proteins of interest or of antibody subunits. The methods and systems described throughout this disclosure with respect to antibody fragments may also be applied to intact proteins of interest, for example a sample of a protein of interest that has not been contacted to a proteolytic enzyme, and/or to antibody subunits, for example a sample of an antibody of interest that has been contacted to a proteolytic enzyme capable of generating antibody subunits, such as IdeS or papain.

In embodiments, the capillary may include a separation matrix, which can be added in an automated fashion by the apparatus and/or system. In some embodiments, the sample is loaded onto a stacker matrix prior to separation. The separation matrix, in one embodiment, is a size separation matrix, and has similar or substantially the same properties of a polymeric gel, used in conventional electrophoresis techniques. Capillary electrophoresis in the separation matrix is analogous to separation in a polymeric gel, such as a polyacrylamide gel or an agarose gel, where molecules are separated on the basis of the size of the molecules in the sample, by providing a porous passageway through which the molecules can travel. The separation matrix permits the separation of molecules by molecular size because larger molecules will travel more slowly through the matrix than smaller molecules. In some embodiments, the one or more capillaries comprise a separation matrix. In some embodiments, the sample containing an antibody of interest is separated or resolved based on molecular weight. In some embodiments, the separation matrix comprises a sieving matrix configured to separate proteins by molecular weight. In some embodiments, protein components of a sample are separated by molecular weight and the method is a method of detecting and/or discriminating between size variants of an antibody of interest. In some embodiments, antibody fragments of a sample are separated by molecular weight and the method is a method of detecting and/or discriminating between size variants of a contaminating protein of interest.

A wide variety of solid phase substrates are known in the art, for example gels, such as polyacrylamide gel. In some embodiments, resolving one or more proteins of interest includes electrophoresis of a sample in a polymeric gel. Electrophoresis in a polymeric gel, such as a polyacrylamide gel or an agarose gel separates molecules on the basis of the molecule's size. A polymeric gel provides a porous passageway through which the molecules can travel. Polymeric gels permit the separation of molecules by molecular size because larger molecules will travel more slowly through the gel than smaller molecules.

In some embodiments, the sample containing a protein of interest is separated or resolved based on the charge of the components of the sample. In some embodiments, protein components of a sample are separated by charge and the method is a method of detecting and/or discriminating between charge variants of a monoclonal antibody of interest. In some embodiments, fragments of a sample are separated by charge and the method is a method of detecting and/or discriminating between charge variants of an antibody of interest of interest.

In some embodiments, an internal standard can be a purified form of the antibody of interest itself or fragment thereof, which is generally made distinguishable from the antibody of interest in some way. Methods of obtaining a purified form of the antibody of interest itself or fragment thereof can include, but are not limited to, purification from nature, purification from organisms grown in the laboratory (e.g., via chemical synthesis), and/or the like. The distinguishing characteristic of an internal standard can be any suitable change that can include, but is not limited to, dye labeling, radiolabeling, or modifying the mobility of the standard during the electrophoretic separation so that it is separated from the antibody of interest. For example, a standard can contain a modification of the antibody of interest itself or fragment thereof that changes the charge, mass, and/or length (e.g., via deletion, fusion, and/or chemical modification) of the standard relative to the antibody of interest itself or fragment thereof. Thus, the antibody of interest itself or fragment thereof and the internal standard can each be labeled with fluorescent dyes that are each detectable at discrete emission wavelengths, thereby allowing the protein of interest and the standard to be independently detectable. In some instances, an internal standard is different from the antibody of interest itself or fragment thereof but behaves in a way similar to or the same as the antibody of interest itself or fragment thereof, enabling relevant comparative measurements.

As will be appreciated by those of skill in the art, virtually any method of loading the sample in the capillary may be performed. For example, the sample can be loaded into one end of the capillary. In some embodiments, the sample is loaded into one end of the capillary by hydrodynamic flow. For example, in embodiments wherein the fluid path is a capillary, the sample can be loaded into one end of the capillary by hydrodynamic flow, such that the capillary is used as a micropipette. In some embodiments, the sample can be loaded into the capillary by electrophoresis, for example, when the capillary is gel filled and therefore more resistant to hydrodynamic flow.

The capillary can include any structure that allows liquid or dissolved molecules to flow. Thus, the capillary can include any structure known in the art, so long as it is compatible with the methods. In some embodiments, the capillary is a bore or channel through which a liquid or dissolved molecule can flow. In some embodiments, the capillary is a passage in a permeable material in which liquids or dissolved molecules can flow.

The capillary includes any material that allows the detection of the protein of interest within the capillary. The capillary includes any convenient material, such as glass, plastic, silicon, fused silica, gel, or the like. In some embodiments, the method employs a plurality of capillaries. A plurality of capillaries enables multiple samples to be analyzed simultaneously.

The capillary can vary as to dimensions, width, depth and cross-section, as well as shape, for example being rounded, trapezoidal, rectangular, etc. The capillary can be straight, rounded, serpentine, or the like. As described below, the length of the fluid path depends in part on factors such as sample size and the extent of sample separation required to resolve the protein of interest.

In some embodiments, the capillary includes a tube with a bore. In some embodiments, the method employs a plurality of capillaries. Suitable sizes include, but are not limited to, capillaries having internal diameters of about 10 to about 1000 μm, although more typically capillaries having internal diameters of about 25 to about 400 μm can be utilized. Smaller diameter capillaries use relatively low sample loads while the use of relatively large bore capillaries allows relatively high sample loads and can result in improved signal detection.

The capillaries can have varying lengths. Suitable lengths include, but are not limited to, capillaries of about 1 to 20 cm in length, although somewhat shorter and longer capillaries can be used. In some embodiments, the capillary is about 1, 2, 3, 4, 5, or 6 cm in length. Longer capillaries typically result in better separations and improved resolution of complex mixtures. Longer capillaries can be of particular use in resolving low abundance proteins of interest.

Generally, the capillaries are composed of fused silica, although plastic capillaries and PYREX (i.e., amorphous glass) can be utilized. As noted above, the capillaries do not need to have a round or tubular shape. Other shapes, so long as they are compatible with the methods described herein, may also be used.

In some embodiments, the capillary can be a channel. In some embodiments, the method employs a plurality of channels. In some embodiments, the capillary can be a channel in a microfluidic device. Microfluidics employ channels in a substrate to perform a wide variety of operations. The microfluidic devices can include one or a plurality of channels contoured into a surface of a substrate. The microfluidic device can be obtained from a solid inert substrate, and in some embodiments in the form of a chip. The dimensions of the microfluidic device are not critical, but in some embodiments the dimensions are on the order of about 100 μm to about 5 mm thick and approximately about 1 centimeter to about 20 centimeters on a side. Suitable sizes include, but are not limited to, channels having a depth of about 5 μm to about 200 μm, although more typically having a depth of about 20 μm to about 50 μm can be utilized. Smaller channels, such as micro or nanochannels can also be used, so long as they are compatible with the methods. The microfluidic device may comprise an integrated electrospray ionization (ESI) emitter, allowing for direct coupling to a mass spectrometer.

The antibody fragments may be obtained from an antibody of interest, such as a monoclonal antibody. The antibody fragments may be prepared by reduction, enzymatic digestion, denaturation, fragmentation, chemical cleavage or a combination thereof. The methods disclosed herein are applicable to any antibody isotype, such as IgG1, IgG2, IgG3, IgG4, or mixed isotype.

In some embodiments, the methods may further comprise identifying, quantifying, or characterizing charge variants or post-translationally modified variants of a protein of interest or fragment thereof, for example using the UV signal from the peptide portion of the charge variant or post-translationally modified variant or fragment thereof. This may be done for fractions of a sample and allows the selection of specific fractions for further analysis, for example mass spectrometry (MS) analysis. Thus, in further embodiments, the detection step may comprise mass spectrometry or liquid chromatography-mass spectrometry (LC-MS). In applications of mass spectrometry for the analysis of biomolecules, the molecules are transferred from the liquid or solid phases to gas phase and to vacuum phase. Since many biomolecules are both large and fragile (proteins being a prime example), two of the most effective methods for their transfer to the vacuum phase are matrix-assisted laser desorption ionization (MALDI) or electrospray ionization (ESI). Aspects of the use of these methods, and sample preparation requirements, are known to those of ordinary skill in the art. In general, ESI is more sensitive, while MALDI is faster. Significantly, some peptides ionize better in MALDI mode than ESI, and vice versa (Genome Technology, June 220, p 52).

ESI is performed by mixing the sample with volatile acid and organic solvent and infusing it through a conductive needle charged with high voltage. The charged droplets that are sprayed (or ejected) from the needle end are directed into the mass spectrometer, and are dried up by heat and vacuum as they fly in. After the drops dry, the remaining charged molecules are directed by electromagnetic lenses into the mass detector and mass analyzed. In one embodiment, the eluted sample is deposited directly from the capillary into an electrospray nozzle, e.g., the capillary functions as the sample loader. In another embodiment, the capillary itself functions as both the extraction device and the electrospray nozzle. The CE device of the present invention may comprise an integrated ESI emitter.

For MALDI, the analyte molecules (e.g., proteins) are deposited on metal targets and co-crystallized with an organic matrix. The samples are dried and inserted into the mass spectrometer, and typically analyzed via time-of-flight (TOF) detection. In one embodiment, the eluted sample is deposited directly from the capillary onto the metal target, e.g., the capillary itself functions as the sample loader. In one embodiment, the extracted analyte is deposited on a MALDI target, a MALDI ionization matrix is added, and the sample is ionized and analyzed, e.g., by TOF detection.

In some embodiments, other ionization modes are used, e.g. ESI-MS, turbospray ionization mass spectrometry, nanospray ionization mass spectrometry, thermospray ionization mass spectrometry, sonic spray ionization mass spectrometry, SELDI-MS and MALDI-MS. In general, an advantage of these methods is that they allow for the “just-in-time” purification of sample and direct introduction into the ionizing environment. It is important to note that the various ionization and detection modes introduce their own constraints on the nature of the desorption solution used, and it is important that the desorption solution be compatible with both. For example, the sample matrix in many applications must have low ionic strength, or reside within a particular pH range, etc. In ESI, salt in the sample can prevent detection by lowering the ionization or by clogging the nozzle. This problem is addressed by presenting the analyte in low salt and/or by the use of a volatile salt. In the case of MALDI, the analyte should be in a solvent compatible with spotting on the target and with the ionization matrix employed. In embodiments, the method further includes identifying the antibody fragments, for example the sequence of the antibody fragments. In embodiments, the method further includes identifying the post-translational modification present on the antibody fragments. In embodiments, the method further includes post-translational modification profiling of the antibody of interest. In embodiments, the method further includes post-translational modification mapping of post-translational modification hotspots by reduced peptide mapping LC-MS/MS analysis.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

The following examples are provided to illustrate particular features of certain embodiments. However, the particular features described below should not be considered as limitations on the scope of the invention, but rather as examples from which equivalents will be recognized by those of ordinary skill in the art.

### EXAMPLES

The following examples are put forth so as to provide those of ordinary skill in the art with a complete disclosure and description of how to make and use the methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Materials and Methods for Examples 1-6

Reagents and Materials. Deionized water was generated with a Milli-Q integral 10 water purification system with a MilliPak Express 20 filter (MilliporeSigma, Burlington, MA). NISTmAb (Reference material 8671, humanized IgG1_{K} monoclonal antibody) was purchased from the National Institute of Standards and Technology (Gaithersburg, MD). All other antibodies including mAb1-7 and bsAb1-3 were generated internally by Regeneron Pharmaceuticals, Inc. (Tarrytown, NY). Dimethyl sulfoxide (DMSO, HPLC grade) was purchased from Thermo Fisher Scientific (Rockford, IL). Urea (BioXtra) was purchased from MilliporeSigma. ProteinSimple methyl cellulose (MC) solution at 0.5% and 1%, Pharmalytes (broad range pH 3-10), and pI markers were purchased from VWR International (Radnor, PA). ZipChip HRN chips with a 22 cm separation channel and a native assay kit with Native Antibodies background electrolyte (BGE) were acquired from 908 Devices (Boston, MA).

Sample preparation. To remove N-glycans from each heavy chain C_{H}2 domain, NISTmAb sample was treated with PNGase F (1 IUB milliunit per 10 μg of protein) at 45° C. in Milli-Q water for 1 hour.

For bsAb impurity analysis, the concentration of each bsAb stock solution and two homospecific mAb impurities (referred to as HC/HC and HC*/HC*) were measured based on the UV absorbance at 280 nm and the corresponding extinction coefficient. A series of solutions were prepared through mixture of bsAb with percentages of homospecific mAb impurities at 0.2% and 1.0%.

Forced degradation study. NIST IgG1 mAb (5 mg/mL, pH 6.0) was incubated at 45° C. for 0, 1, 4, 8, 15 and 28 days.

IdeS treatment. For subunit analysis, each NISTmAb sample was diluted to 2 mg/mL with Milli-Q water. Then 125 units of IdeS (Promega) was added to 100 μg of mAb at enzyme/antibody ratio of 1.25/1. The mixture was incubated at 37° C. with shaking at 600 rpm for 30 minutes to generate F(ab′)2 and Fc fragments. Control and stressed samples after each study/treatment were stored immediately at −20° C. A summary of the method is shown in FIG.

Native microfluidic CE-MS (nCE-MS). The intact mass analysis of antibodies and charge variants was conducted with the ZipChip CE interface (908 Devices, Boston, MA) coupled to an Exactive Plus EMR Orbitrap mass spectrometer (Thermo Scientific, Bremen, Germany). Samples were loaded on the sample well of a microfluidic chip by a model 840 autosampler with a pressure assistance start time at 0.2 min and a replicate delay of 30 seconds. The injection volume was 1 nL. Charge variants were separated in the separation channel of the microfluidic chip by using Native Antibodies BGE, 3.8% DMSO, pH ˜5.5, under a field strength of 650 V/cm for 15 min. Each separated charge variant species was then ionized and subjected to MS analysis. MS data acquisition was performed through a full scan at a resolution of 17,500 in positive mode with a scan range of 1000-10000 m/z, in-source CID at 100 eV, AGC at 3e6, maximum injection time of 50 ms, and 3 microscans. The ESI parameters were set with spray voltage at 0, capillary temperature at 300° C., S-lens at 150, sheath gas at 2, auxiliary gas at 0, and trapping gas at 1.

Strong cation exchange chromatography. Antibody charge variants were separated on a MabPac SCX-10 column (4×250 mm) and an Agilent 1290 Infinity HPLC. The pH gradient was optimized according to the tested antibody's theoretical pI value. Mobile phase A (MPA) and mobile phase B (MPB) were CX-1 pH gradient buffer A and B with pH 5.6 and 10.2 (Thermo Fisher Scientific), respectively. Briefly, for IgG4 and bispecific IgG4 antibodies, the charge variants were separated with a pH gradient from 5% to 15% MPB in 30 min. For IgG1 antibodies, the pH gradient was adjusted from 45% to 70% MPB in 30 min. Absorbance was measured at a UV wavelength of 280 nm. The flow rate was set to 0.8 mL/min, and the column temperature was set to 25° C.

Imaged capillary isoelectric focusing. The final sample solution contained 0.5 mg/mL protein, 4% (v/v) pH 3-10 Pharmalytes, 2 M urea, and 0.35% (w/v) methylcellulose. Samples were prepared in triplicate in the presence or absence of pI markers. Each sample was hydrodynamically injected into a fluorocarbon-coated silica capillary (100 mm inner diameter, 50 mm observable window, ProteinSimple, San Jose, CA) at 2 bar for 70 seconds with a ProteinSimple iCE3 system. Samples were subsequently pre-focused for 1 minute at 1.5 kV, then subjected to a 7.0 minute focusing period at 3 kV with 100 mM sodium hydroxide as the catholyte and 80 mM phosphoric acid as the anolyte, both in 0.1% (w/v) methyl cellulose. Proteins were monitored during focusing according to the UV absorbance at 280 nm with a scanning charge-coupled device (CCD) camera. Electropherograms were generated with CCD detection of UV light absorption across the separation capillary.

Data analysis. Raw MS spectra were analyzed in Thermo Xcalibur 4.3.73.11 and deconvoluted with Intact Mass™ software version 3.11-1 (Protein Metrics Inc., Cupertino, CA) for charge variant analysis. Base peak chromatograms (BPCs) were processed with Microsoft Excel macros for the same mAb experiment. For data acquired with an Agilent HPLC system, the data were analyzed in OpenLAB CDS ChemStation Edition Rev. C.01.07 SR2.

### Example 1. Investigation of Sensitivity of nCE-MS for Therapeutic Antibodies

This disclosure sets forth the development of a high-resolution, high-sensitivity and high-throughput native capillary electrophoresis (CE)-mass spectrometry (MS) method for antibody charge heterogeneity analysis. A panel of fifteen antibodies, including IgG1, IgG4 and bispecific mAbs, were analyzed using the Zipchip nCE-MS method.

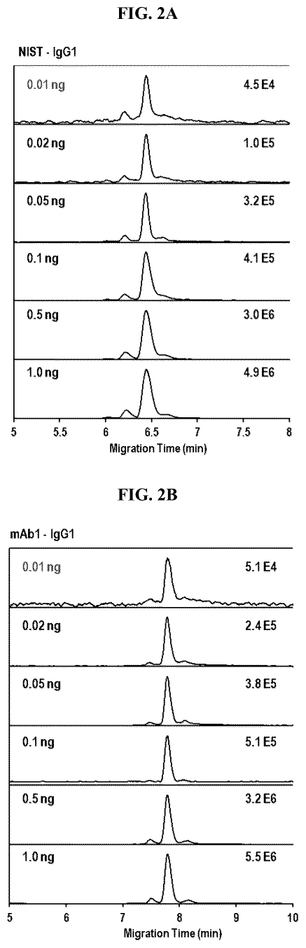

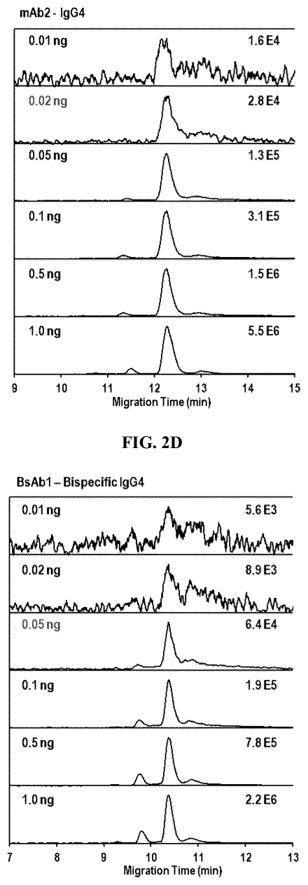

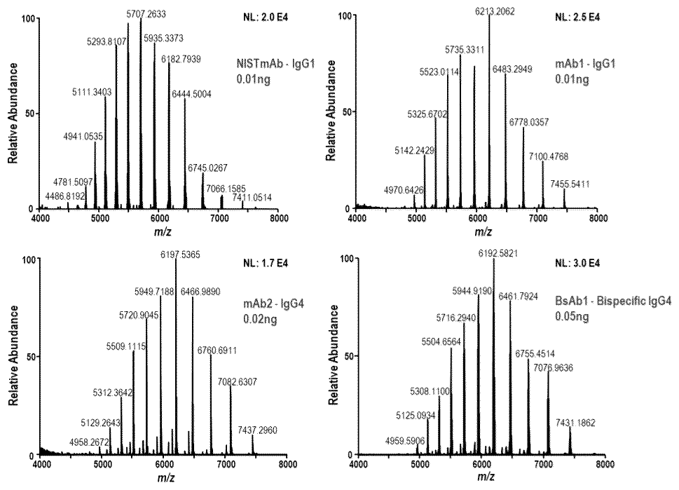

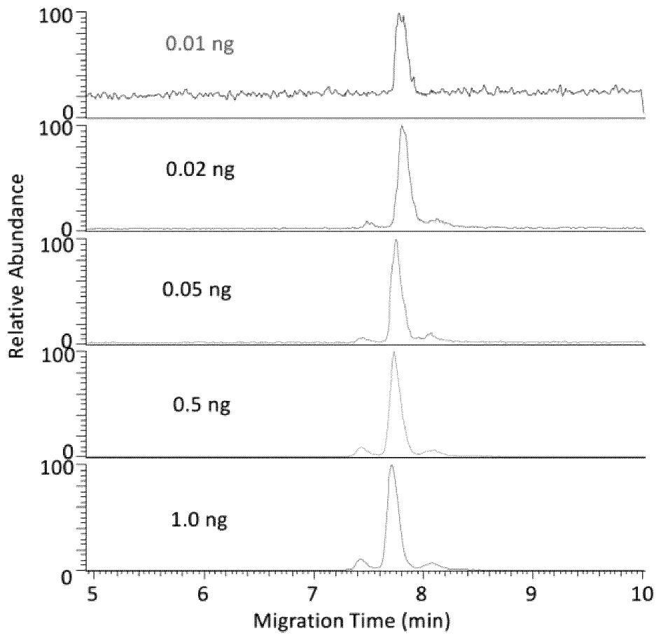

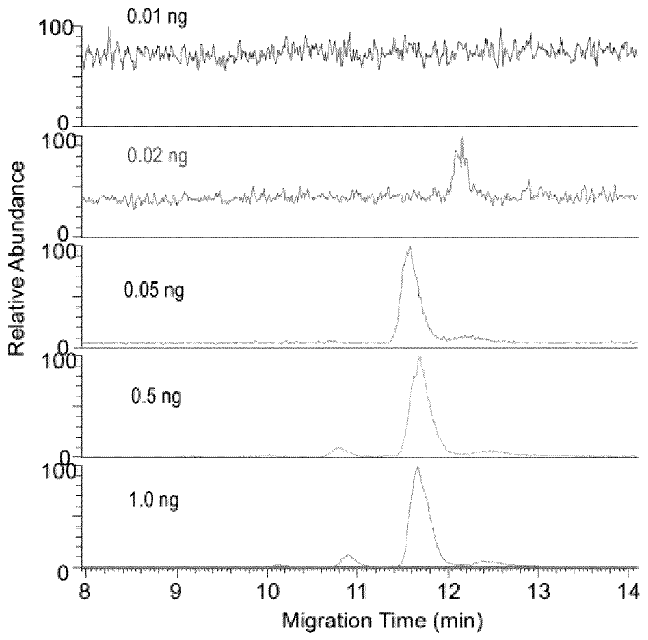

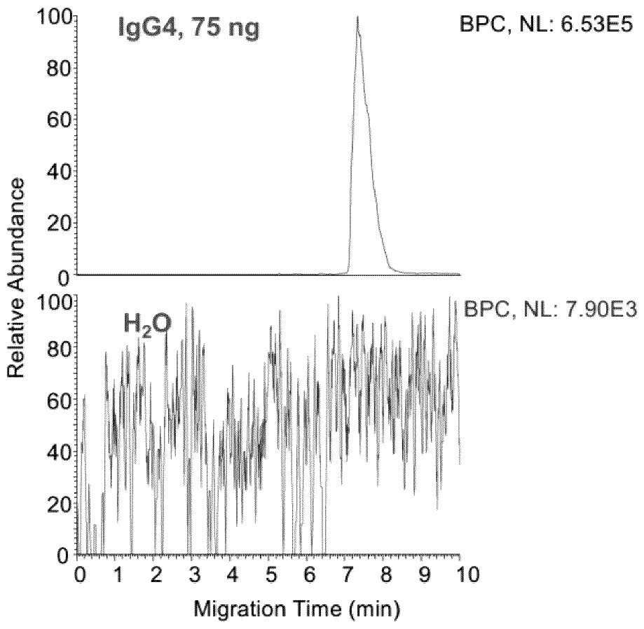

ZipChip nCE-MS combined with nanospray ESI provides ultra-high sensitivity in detecting low abundant charge variant species. The method sensitivity of nCE-MS in analyzing various types of IgG was evaluated by separate injection of different amounts of antibody from 0.01 ng to 1 ng on the chip. The antibody charge separation profile was extracted with BPCs with mass range from 4000 to 9000 m/z, as shown in FIG. 2. In the case of IgG1 antibodies for both NISTmAb and mAb1, a limit of detection (LOD) of 0.01 ng was achieved, as shown in FIG. 2A, FIG. 2B, and FIG. 4A. For IgG4, mAb2 and bsAb1, the MS signal was detected down to 0.02 ng and 0.05 ng, respectively, as shown in FIG. 2C, FIG. 2D, and FIG. 4B. At the LOD for all four mAbs including IgG1, IgG4, and bispecific IgG4, the mass spectra of the main species of each antibody showed a high signal-to-noise ratio, thus demonstrating the excellent sensitivity of the nCE-MS method, as shown in FIG. 3. Moreover, the charge variant profile was directly observed at the LOD for all four mAbs. No run-to-run injection carryover was observed for the nCE-MS method, as shown in FIG. 4C.

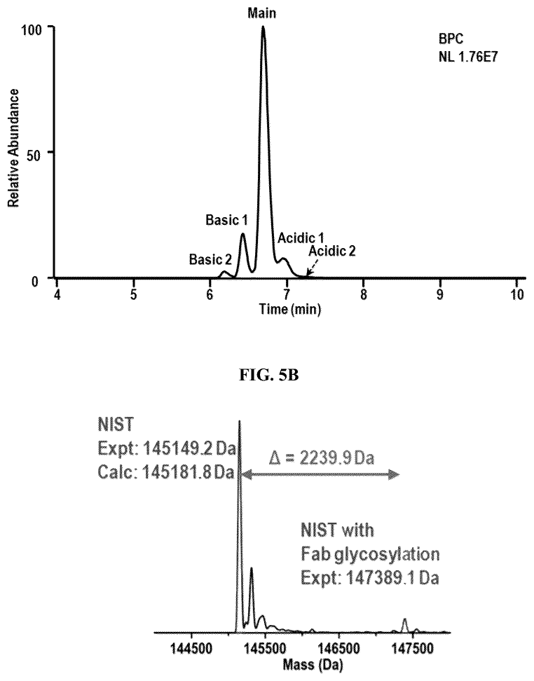

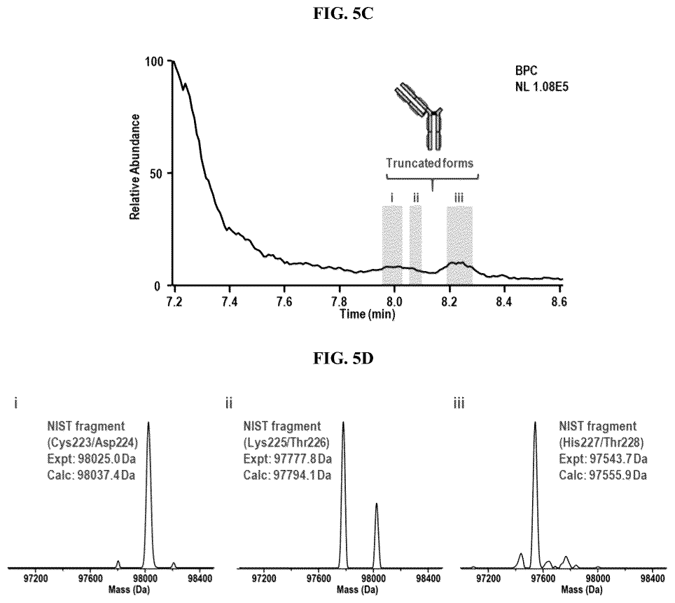

The high sensitivity of the nCE-MS method was also demonstrated by the observation of truncation species with low amounts of sample injection. A deglycosylation reaction step removed all N-glycans attached to the mAb, thus decreasing the proteoform complexity and increasing the sensitivity to detect sequence variations. The electropherogram of deglycosylated NISTmAb showed identical charge variant profile and identifications to those of intact NISTmAb, as shown in FIG. 2A and FIG. 5A. In the A2 acidic variant, Fab glycosylation was detected with a 2 ng injection, as shown in FIG. 5B (Yan et al., 2018). Additionally, three truncation species on the upper hinge region were detected including the cleavage at Cys223/Asp224, Lys225/Thr226, and His227/Thr228, as shown in FIGS. 5C and 5D, thus demonstrating the high sensitivity of the nCE-MS method (Yan et al., 2018).

In order to assess the sensitivity of the method for additional fragmented and truncated species, a forced degradation study and subunit digestion study was conducted using NISTmAb. The NISTmAb reference standard and its heat-stressed forms were analyzed following incubation at 45° C. for up to 28 days. Samples with different incubation times were cleaved by IdeS digestion to generate F(ab′)2 or Fc associated subunit species. The intact mass analysis of both control and stressed antibodies was conducted using a universal Zipchip nCE-MS method. The PTM hotspots were identified by reduced peptide mapping LC-MS/MS analysis to elucidate the elevated charge variants under stressed conditions. Intact mass data was processed by PMi-Intact software. The elevated charge variants were allocated to F(ab′)₂ or Fc by the subunit charge variant analysis. Reduced peptide mapping data was processed by BioPharma Finder 3.0 and Skyline-daily 4.2 for PTMs identification and quantification, respectively.

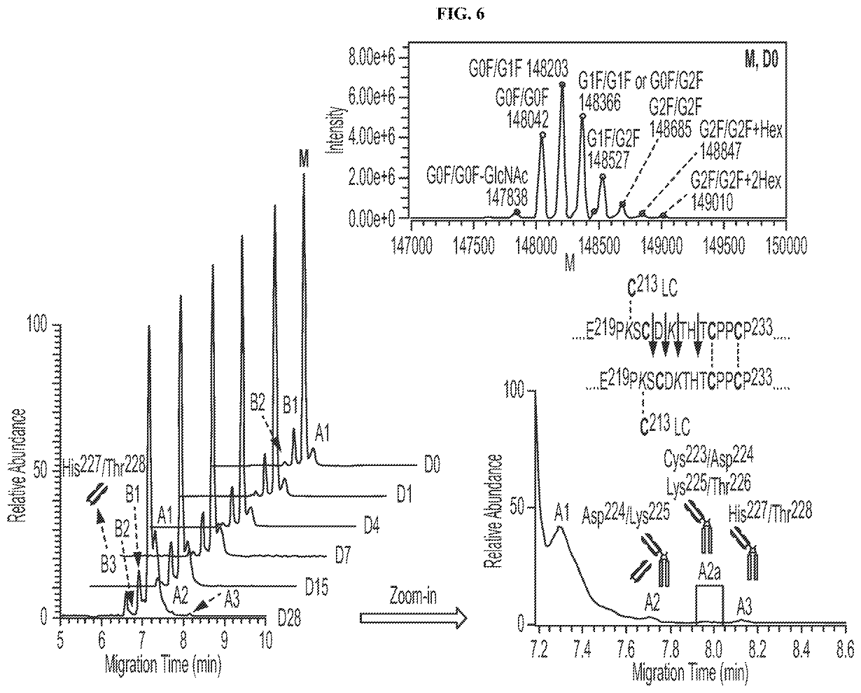

Deconvoluted mass data indicated that 4 major Fab cleavages at the upper hinge region of NISTmAb were generated upon 28-day heat stress, in addition to significantly increased acidic variant (A1) compared to the control (FIG. 6 and Table 2).

TABLE 2
Summary of charge variant identification

| Charge | Control | Stressed NISTmAb |
|---|---|---|
| Variants | NISTmAb | (45° C., pH 6.0, 28 days) |
| Basic 1 (B1) | +1 C-terminal | +1 C-terminal lysine |
| | lysine | |
| Basic 2 (B2) | +2 C-terminal | +2 C-terminal lysine |
| | lysine | |
| Basic 3 (B3) | ND | Fab cleavage at His²²⁷/Thr²²⁸ |
| Acidic 1 (A1) | +Deamidation | +Deamidation |
| Acidic 2 (A2) | ND | Fab cleavage at Asp²²⁴/Lys²²⁵ |
| Acidic 2a (A2a) | ND | Loss of Fab cleaved at |
| | | Cys²²³/Asp²²⁴ and |
| | | Lys²²⁵/Thr²²⁶ |
| Acidic 3 (A3) | ND | Loss of Fab cleaved at His²²⁷/Thr²²⁸ |

Note:
ND—not detected

Acidic and basic charge variants were well separated from the main peak for the NIST antibody standard by Zipchip CE running at native conditions. The MS analysis identified two basic variants corresponding to the antibody with 1 and 2 unprocessed C-terminal lysines on the heavy chain, while an acidic variant was mainly caused by deamidation.

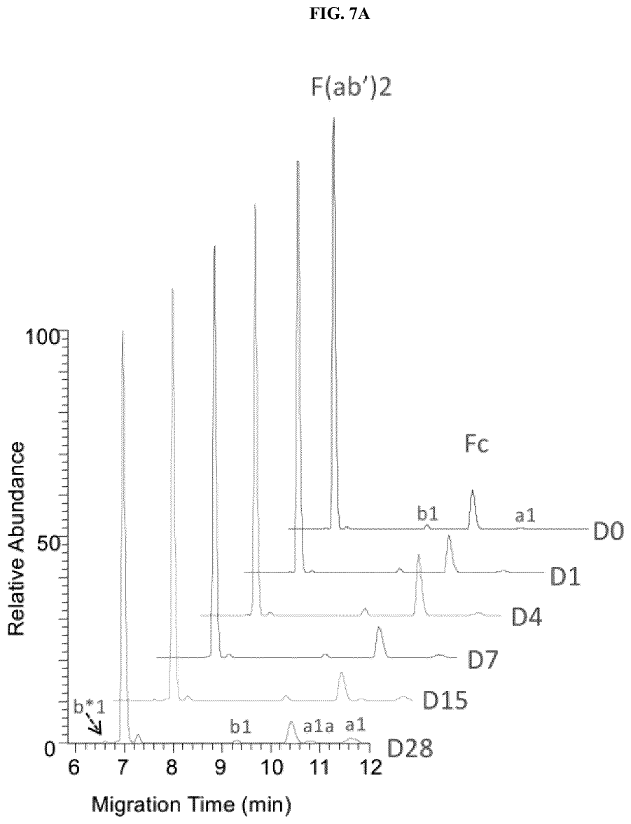

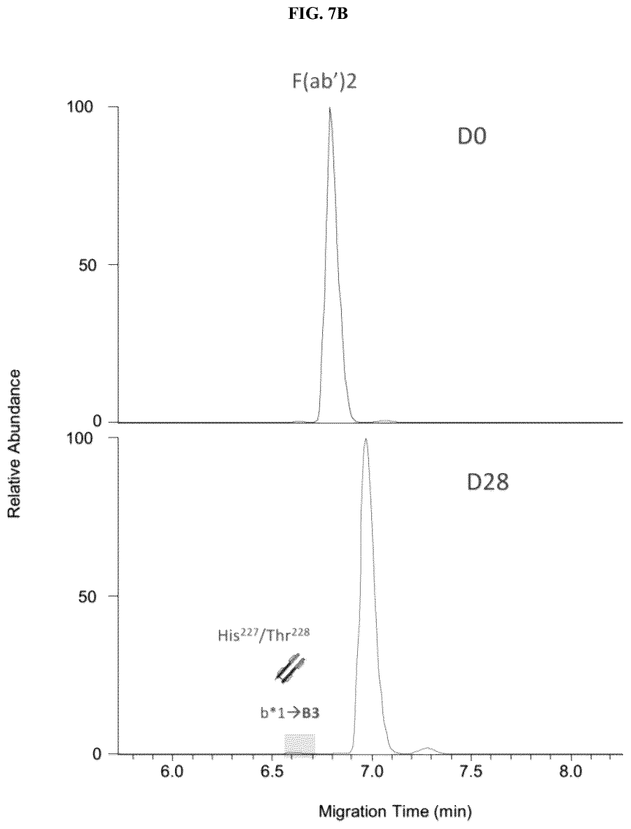

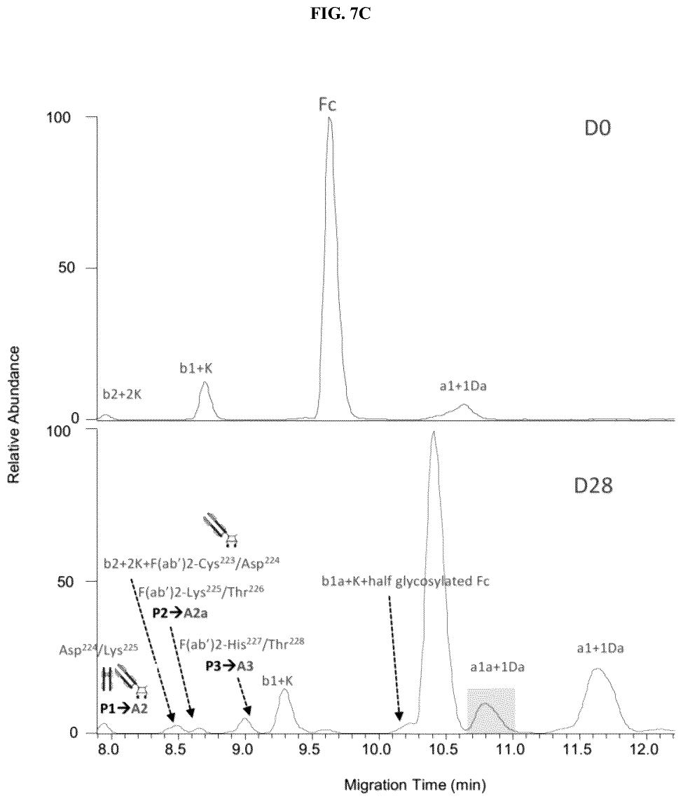

F(ab′)2 and Fc were well separated by the universal nCE-MS method. All minor peaks were identified, as shown in FIGS. 7A-7C. New charge variants resulting from 28-day incubation are indicated in blue shaded regions. Two basic variants with 1 and 2 unprocessed C-terminal lysines were located in the Fc region (FIG. 7C). The basic variant 3, resulting from Fab cleavage at His^{227‘/Thr}²²⁸ in stressed sample (45° C., D28), was identified as F(ab’)2 basic variant (b*1) in FIG. 7B. All other Fab cleavage sites were found in the acid region of F(ab′)2 and in the same order as those were identified during intact antibody analysis. For Fc acidic region, a new acidic variant A1a caused by Asp isomerization showed up in D28 sample. Compared to main Fc, the +1 Da mass increase of both A1 and Ala indicated that the acidic variant might be caused by deamidation. This was confirmed by peptide mapping, as shown in Table 3.

TABLE 3
Summary of major PTMs showing differences under stressed conditions

| Antibody | | | D 0 | | | | | |
|---|---|---|---|---|---|---|---|---|
| Region | PTMs | Site | (Control) | D 1 | D 4 | D 7 | D 15 | D 28 |
| Fc | Deamidation | HC Asn387 | 1.61% | 1.64% | 1.71% | 1.91% | 2.15% | 2.64% |
| | Deamidation | HC Asn392 | 0.75% | 0.83% | 1.04% | 1.40% | 1.99% | 3.03% |
| | Oxidation | HC Met255 | 1.23% | 1.56% | 1.66% | 1.88% | 2.05% | 3.00% |
| | Isomerization | HC Asp283 | 0.65% | 0.70% | 0.87% | 1.29% | 1.70% | 2.81% |
| F(ab′)2 | Isomerization | LC Asp166 | 2.65% | 3.28% | 3.58% | 4.64% | 5.59% | 7.62% |

### Example 2. Separation of Antibody Mixtures for High-Throughput Intact Mass Analysis

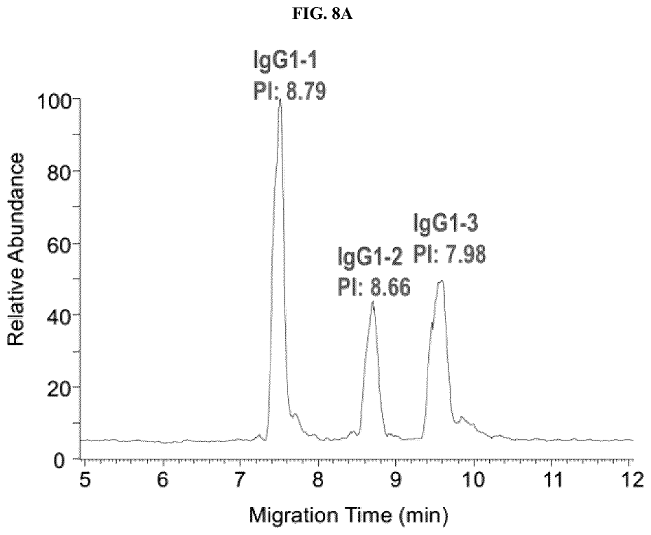

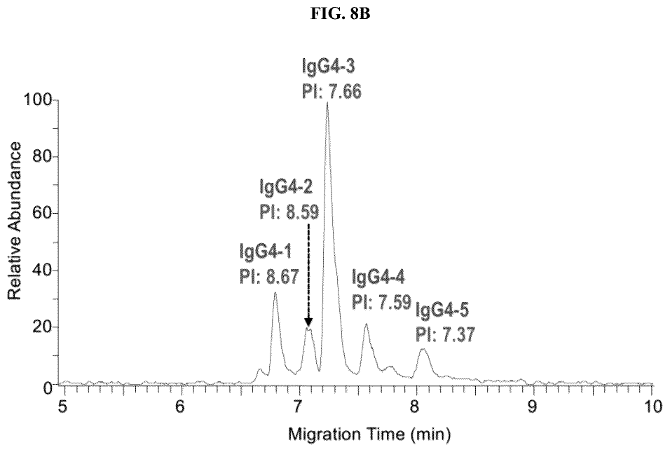

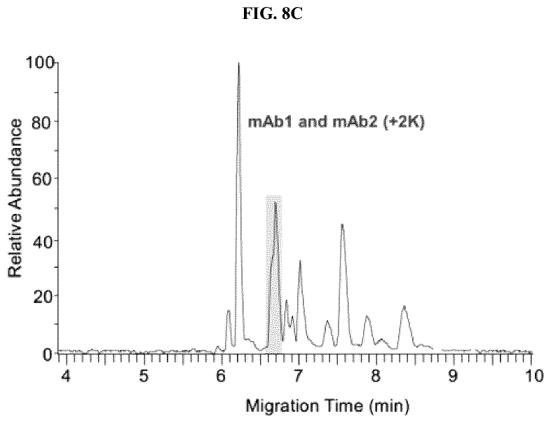

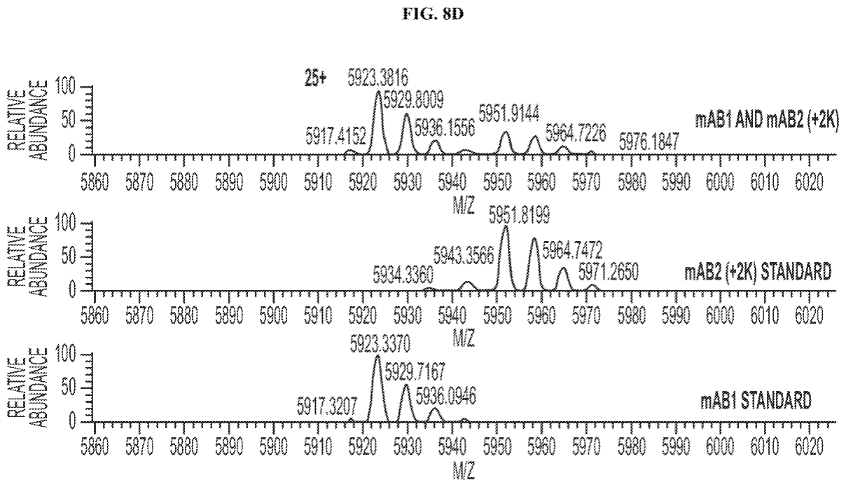

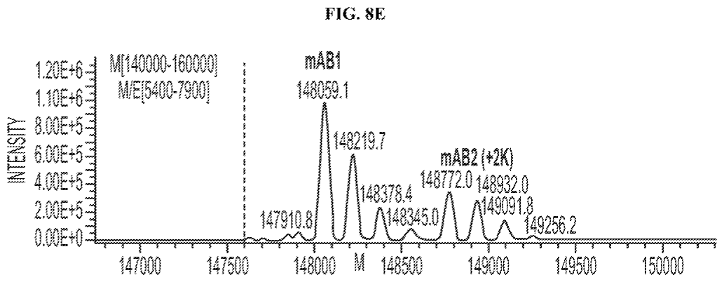

Three IgG1 mAbs in mixture 1 (FIG. 8A) and five IgG4 bispecific mAbs in mixture 2 (FIG. 8B) were well separated by the ZipChip nCE-MS method of the present invention, which can be applied for intact mass analysis of co-formulated drugs. Even if two antibodies co-migrated together (blue shaded area in FIG. 8C), those can be identified individually from native MS data, as shown in FIG. 8D and FIG. 8E. FIG. 8E shows the convoluted spectra. Besides high-resolution charge variant analysis of a single antibody, the ZipChip nCE-MS method can also be used as a high-throughput and high-sensitivity approach for intact mass analysis of antibody mixture and ADCs.

### Example 3. System Robustness

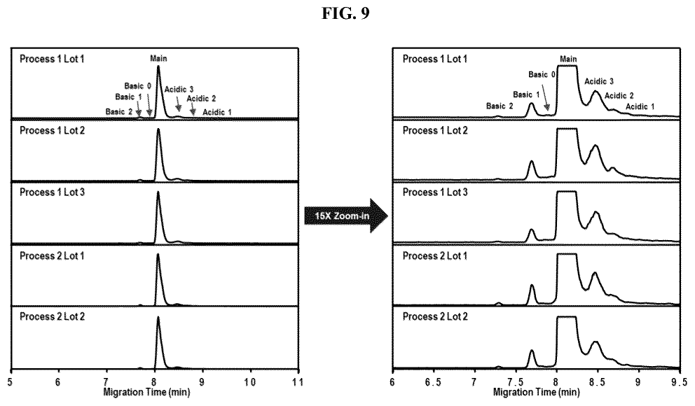

System robustness is important to assess when method is applied in various analytical tasks in industrial settings. This property is particularly crucial for comparability studies such as cell line selection and process optimization. As shown in FIG. 9 for an IgG4 mAb3, three lots from process 1 and two lots from process 2 were subjected to nCE-MS analysis. The charge variant profiles from the nCE-MS analysis of the five samples were highly comparable, showing three basic variants and three acidic variants. Moreover, MS analysis allowed for PTM characterization and the identification of processes related PTM differences, as shown in Table 4.

TABLE 4
Mass table for charge variant characterization of mAb3

| Charge Variant Peaks | Proposed Charge Variant Identity |
|---|---|
| Basic 2 | 2× C-terminal Lys |
| | 2× Gly loss with amidation |
| Basic 1 | 1× C-terminal Lys |
| | 1× Gly loss with amidation |
| Basic 0 | 2× N-terminal pyroglutamate |
| Main | mAb |
| Acidic 3 | Deamidation |
| | Glycation |
| Acidic 2 | Deamidation |
| | Glycation |
| | 1× NeuAc |
| Acidic 1 | Glycation |

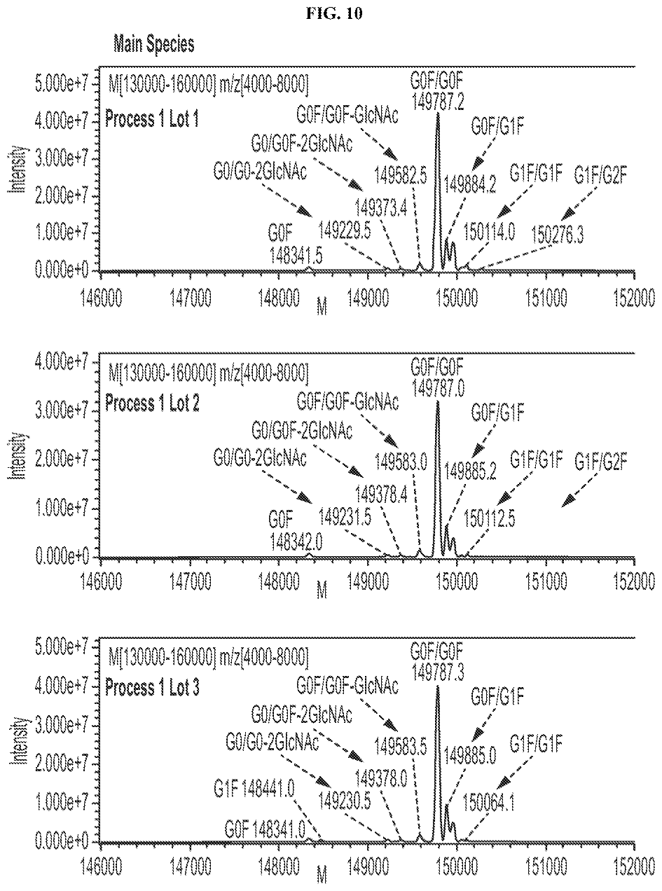

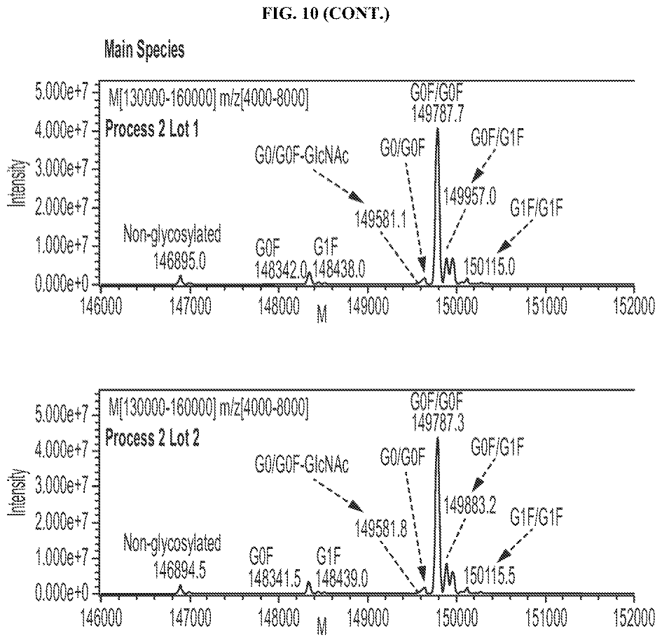

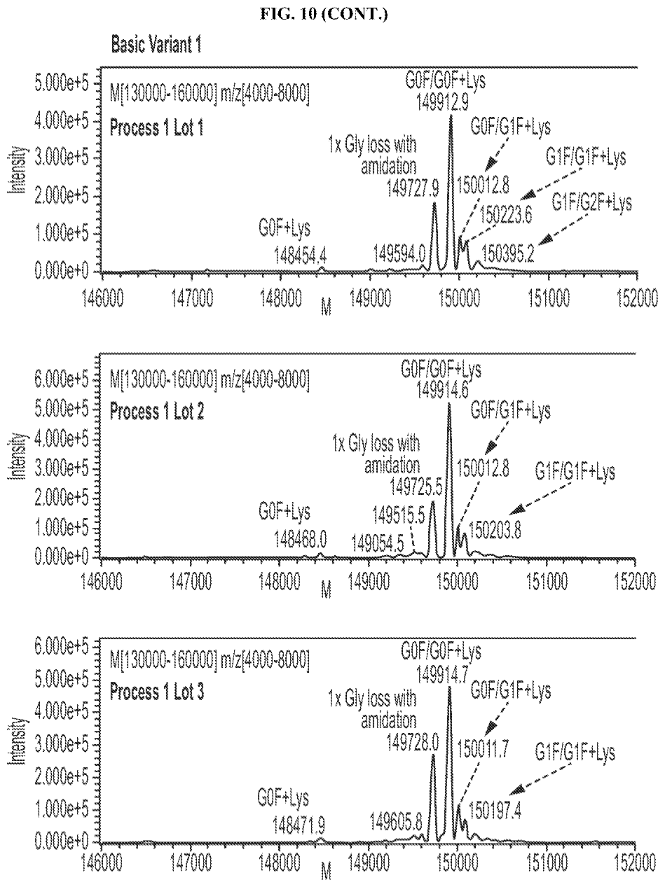

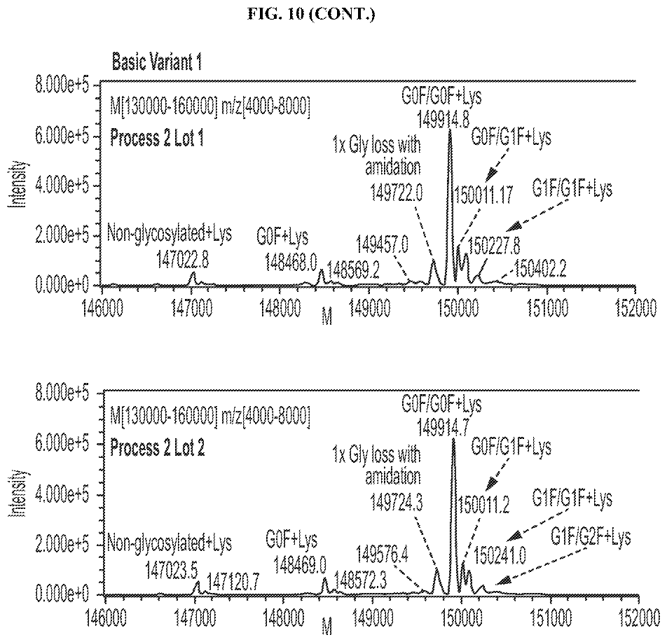

In the main species, more non-glycosylated species were observed in samples from process 2 than process 1, as shown in FIG. 10A. Additionally, higher levels of glycine loss with amidation were observed in samples from process 1 than process 2, as shown in basic variant 1, as shown in FIG. 10B. The highly comparable charge variant profile, in addition to the consistent results among samples from the same process, demonstrated the system robustness of nCE-MS analysis, which is suitable for comparability studies.

### Example 4. Comparison of Antibody Charge Variant Separation Among nCE-MS, SCX-UV, and iCIEF

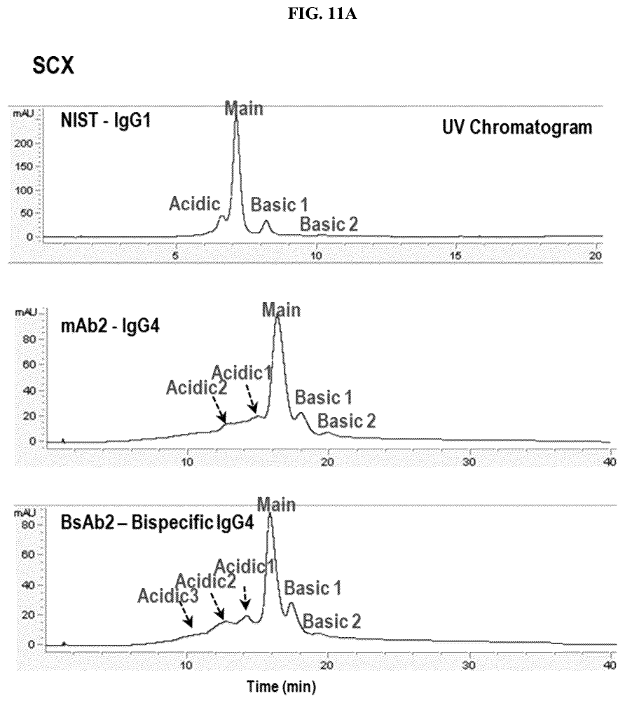

nCE-MS is a new method for charge variant analysis, in contrast to SCX and iCIEF, which are commonly used as characterization and release assays. In this study, the nCE-MS method was first compared to SCX with UV detection. Three types of antibodies including IgG1 NISTmAb, IgG4 mAb2, and bispecific IgG4 bsAb2 were used for this comparison. The charge variant separation profiles of these three types of antibodies are shown in FIG. 11 between the SCX-UV method using a pH gradient and nCE-MS. Comparable high-resolution charge variant separations of all types of IgG were obtained between platforms, including the same number of basic and acidic variants. Compared with SCX-UV, which usually requires injection of more than 10 μg, the nCE-MS method enabled high resolution charge variant separation with only a 1 ng injection. More importantly, each charge variant was directly identified by online MS analysis. In comparison, the SCX-UV method generally uses MS-incompatible salts, and often requires either fraction collection or two-dimensional liquid chromatography coupled with MS to elucidate the PTMs in the charge variants (Alvarez et al., 2011, Anal. Biochem., 419(1):17-25; Jaag et al., 2021, J. Chromatogr. A, 1636:461786).

Recently, SCX coupled to native MS (SCX-nMS) has been used for charge variant analysis of mAbs (Yan et al., 2018; Ma et al.; Haberger et al.). Similar charge variant profiles have been obtained between SCX-nMS and nCE-MS analysis (Fussl et al., 2020, Anal. Chem., 92(7):5421-5438). However, sample amounts of approximately 50 μg are often required for SCX-nMS with post-column flow splitting to enhance sensitivity, and to provide the same detection of truncated species and Fab glycosylation species shown in FIG. 5 (Yan et al., 2018; Ma et al.). In comparison, only a 2 ng injection (with 10 μg transferred to the sample well) was required with the integrated nCE-MS method. Additionally, the unused portion of the protein sample could be recovered from the ZipChip interface after the nCE-MS experiment and subjected to other assays, thus providing experimental flexibility, particularly in the drug discovery stage and other situations in which protein amounts are limited.

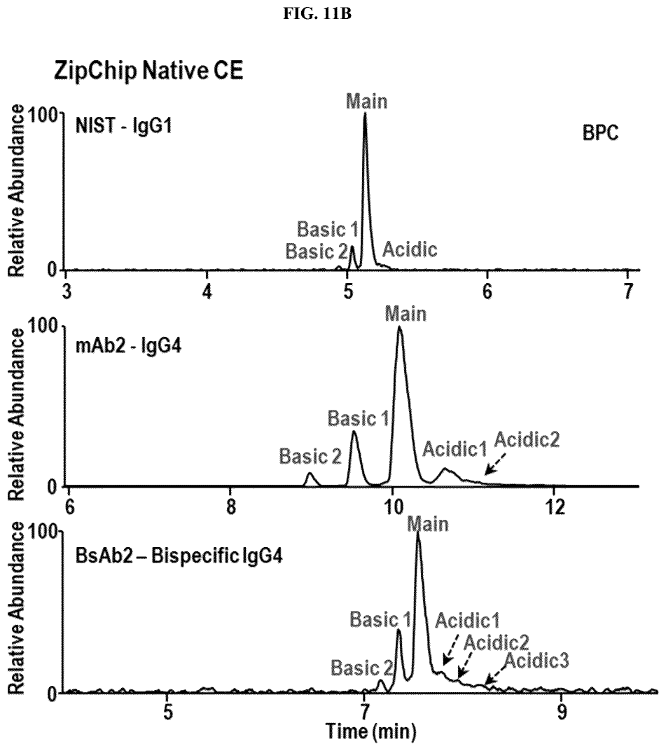

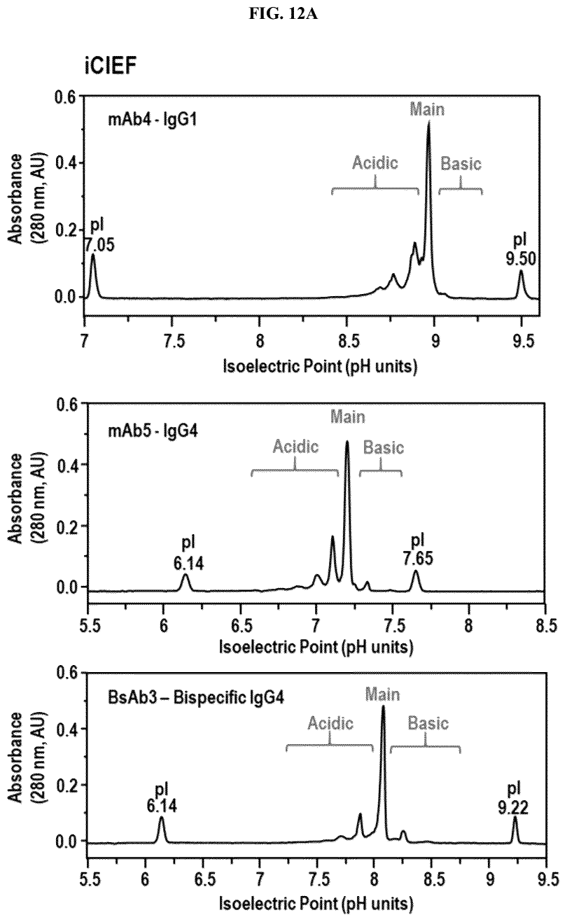

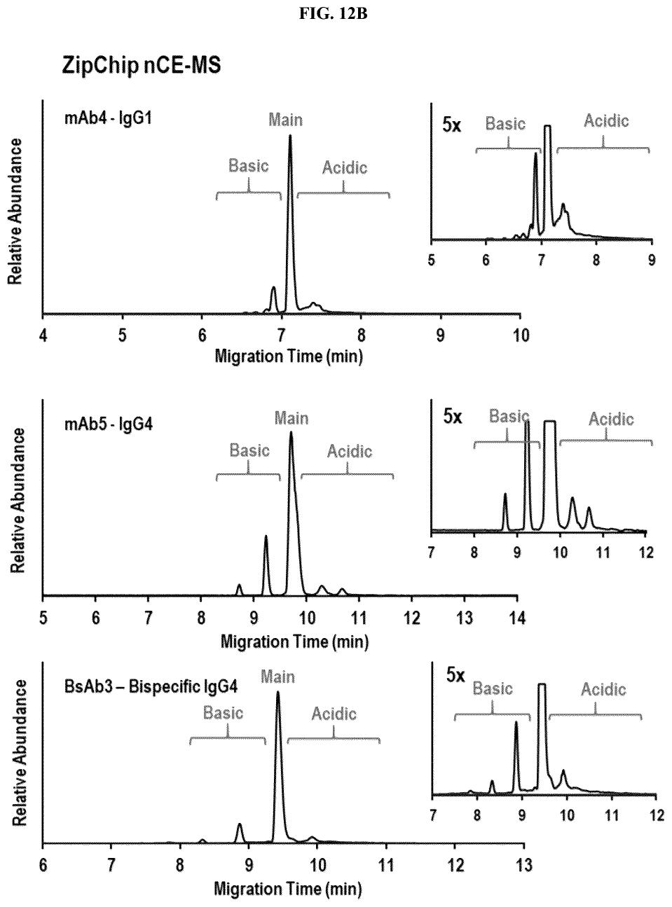

Charge variant separation between iCIEF and nCE-MS was also compared. Three types of antibodies including IgG1 mAb4, IgG4 mAb5, and bispecific IgG4 bsAb3 were used to compare these two methods. As shown in FIG. 12, the charge variants between the iCIEF and nCE-MS analysis showed several differences. In the case of IgG1 mAb4, the number of acidic variants between these two methods differed and the nCE-MS methods showed fewer acidic species than the iCIEF method, as shown in FIG. 12A. In contrast, the basic variant was more clearly observed in the nCE-MS method than the iCIEF method. In the case of both IgG4 mAb5 and bispecific IgG4 bsAb3, comparable electropherograms were observed between these two methods, as shown in FIGS. 12B and 12C. A recent study has suggested that electropherograms between iCIEF and nCE-MS analysis are comparable (Cao et al.). In the examples provided in this study, differences in electropherograms were observed, such as for IgG1 mAb4. The nCE-MS method also showed relatively lower resolution separation in the acidic region than iCIEF analysis, possibly because of tailing from the main species and potential diffusion caused by peak broadening during the long migration time in the separation channel. The differences are likely due to the different nature for separation between these two methods, where iCIEF separates analytes according to intrinsic pI whereas nCE-MS method is based on capillary zone electrophoresis that takes into account both the solvated charge and size of the analytes. Recently, direct coupling of iCIEF and MS has been used to accelerate peak identification for iCIEF analysis; however, the method remains under development, and wide adoption of this technique has yet to occur (Mack et al.; He et al.; Dai et al., 2018, Anal. Chem., 90(3):2246-2254). According to the observations in this study, nCE-MS is a highly sensitive, high-resolution approach for the characterization of antibody charge variants, and the similarity in electropherograms between iCIEF and nCE-MS may aid in the identification of antibody charge variants for iCIEF assays.

Despite the advantages of nCE-MS analysis, iCIEF and SCX assays are performed with UV detection, which can directly enable quantification of charge variants, whereas nCE-MS relies on BPCs from MS and provides the relative quantification. Additionally, the relative quantification is also impacted by the differential ionization efficiency among acidic, main, and basic species. The SCX-nMS method with flow splitting to a UV detector can be used to quantitatively measure the percentages of charge variants (Yan et al., 2018).

### Example 5. Bispecific Antibody Impurity Analysis

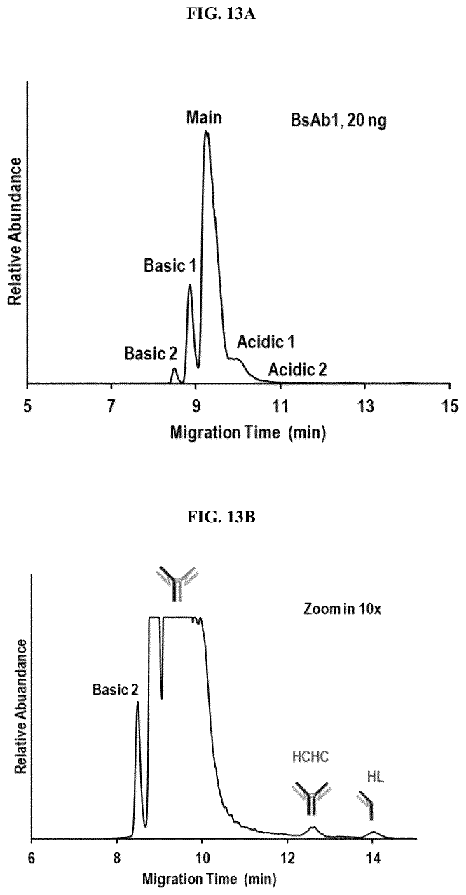

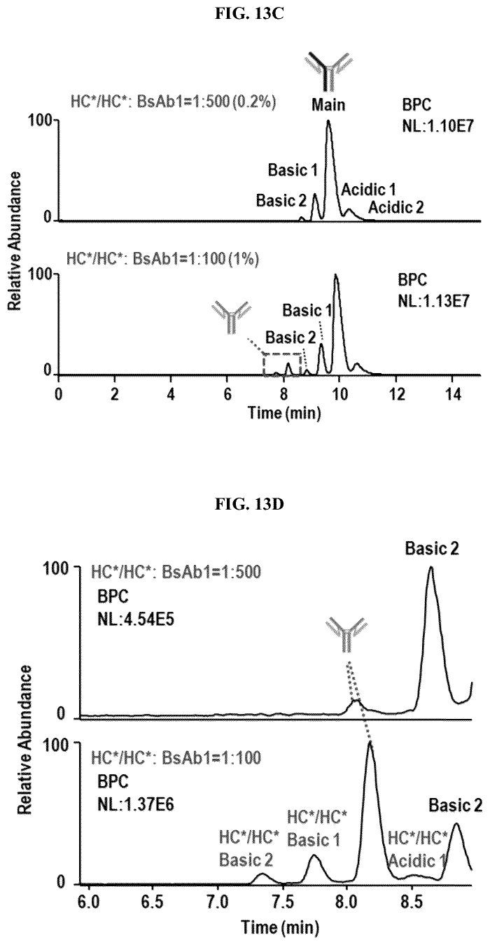

Impurities in bsAb therapeutics are a subject of interest for the pharmaceutical industry (Yan et al., 2019, Anal. Chem., 91(17):11417-11424). In this study, the bsAb of interest comprised two identical light chains and two different heavy chains, HC and HC* (Smith et al., 2015, Sci. Rep., 5(1):17943; Tustian et al., 2016, MAbs, 8(4):828-838). Although homospecific mAb impurities, which are composed of two identical light chains and two identical heavy chains (either HC or HC*), are removed during purification in the manufacturing process, some residual homospecific mAb impurities (referred to as HC/HC and HC*/HC*) remain in the desired bsAb product (Li, 2019, Protein Expr. Purif., 155(112-119). Quantification of these homospecific mAb species is necessary to ensure product safety and efficacy. The nCE-MS method was used for the analysis of the bispecific IgG4 bsAb1, as shown in FIG. 13A. For this bispecific antibody, common charge variants were detected, including antibody with one or two unprocessed C-terminal lysine residues in the basic variants, and deamidation, glycation, as well as one or two N-acetylneuraminic acid residues in the acidic variants, as shown in Table 5.

TABLE 5
Mass table for charge variant characterization of bsAb1

| Charge Variant Peaks | Proposed Charge Variant Identity |
|---|---|
| Basic 2 | 2× C-terminal Lys |
| Basic 1 | 1× C-terminal Lys |
| Main | mAb |
| Acidic 1 | Deamidation |
| | Glycation |
| | 1× NeuAc |
| Acidic 2 | Deamidation |
| | Glycation |
| | 1× NeuAc |
| | 2× NeuAc |
| Others | HC/HC homospecific mAb impurities |
| | HL half antibody |

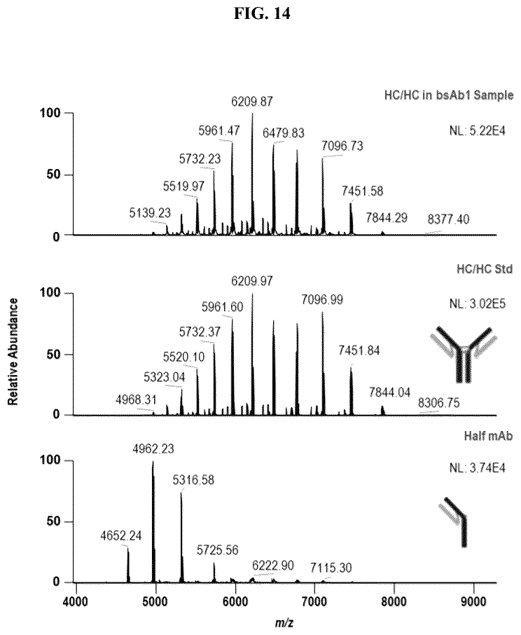

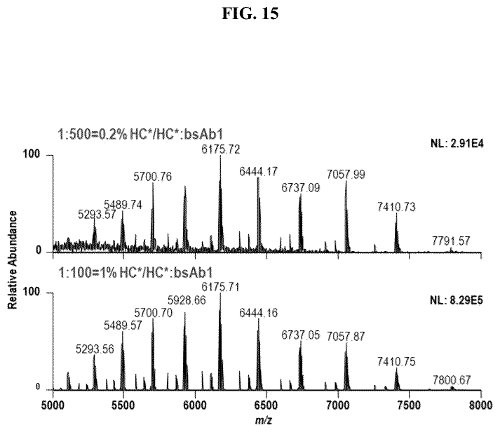

In the zoom-in view, low abundant HC/HC and half antibodies were observed after 20 ng injection into the nCE-MS, thus demonstrating the high sensitivity of the nCE-MS method, as shown in FIG. 13B and FIG. 14. However, HC*/HC* was not detected in the sample. To understand the relative abundance of HC*/HC* impurity in the bsAb1, bsAb1 sample was spiked with HC*/HC* impurity at a ratio of 1:500 (0.2%) and 1:100 (1%) and then analyzed using nCE-MS. Because the pI values of the HC and HC* are different, HC/HC and HC*/HC* impurities and bsAb should be separated in the nCE-MS method. In this spiking experiment, the HC*/HC* impurity was detected at a level of 0.2% in spiked bsAb1 samples, as shown in FIG. 13C and FIG. 15. The high sensitivity of the nCE-MS method was also demonstrated by the observation of separate peaks for charge variants of HC*/HC* impurity at a level of 1%, as shown in FIG. 13D. With this method, the HC*/HC* impurity was determined to be below 0.2%, if any, in the bsAb1 sample.

### Example 6. Charge Heterogeneity Analysis of New Formats of Biotherapeutics

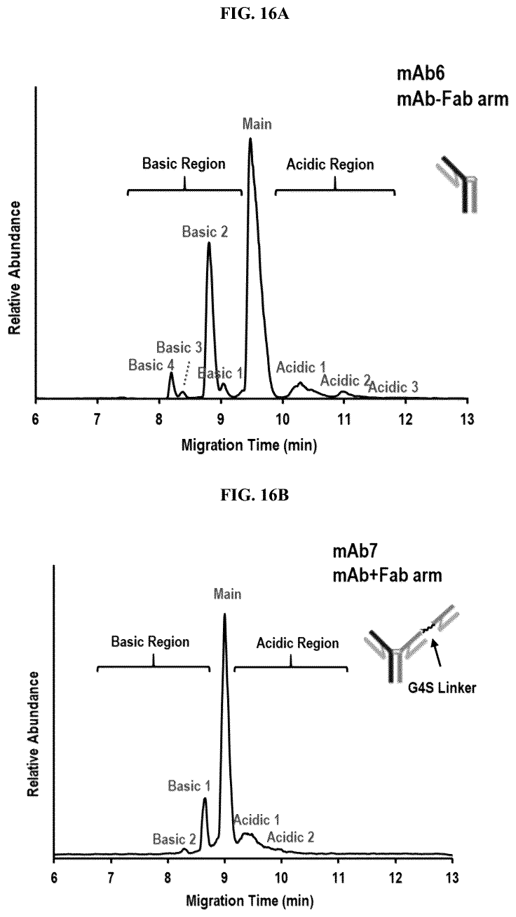

In addition to traditional formats of mAbs, including IgG1, IgG4, and bispecific antibodies described in the preceding sections, nCE-MS can analyze the charge variants of new formats of antibody derivatives. For example, mAb6 is a therapeutic antibody in “N−1” format where one Fab arm is removed, and it has Gln in the N-terminus of the HC. During mAb manufacturing, Gln would be converted into pyroglutamate. However, a small percentage of Gln is often found to be unconverted, thus resulting in a basic charge variant relative to the main species, which is a mAb with complete conversion of Gln to pyroglutamate (Liu et al., 2019, J. Pharm. Sci., 108(10):3194-3200). nCE-MS analysis revealed eight species, including four basic variants and three acidic variants, as shown in FIG. 16A and Table 6.

TABLE 6
Mass table for charge variant characterization
of mAb6 in “N − 1” format

| Charge Variant Peaks | Proposed Charge Variant Identity |
|---|---|
| Basic 4 | 2× C-terminal Lys |
| Basic 3 | 1× C-terminal Lys and 1× unconverted Gln |
| Basic 2 | 1× C-terminal Lys |
| Basic 1 | 1× unconverted Gln |
| Main | mAb |
| Acidic 1 | Deamidation |
| | Glycation |
| | 1× NeuAc |
| Acidic 2 | 1× NeuAc |
| | 2× NeuAc |
| Acidic 3 | 2× NeuAc |
| | 3× NeuAc |

In the basic variants, PTMs including one or two unprocessed C-terminal Lys residues, one unconverted Gln, and a combination of one unprocessed C-terminal Lys residue and one unconverted Gln were observed. Three acidic variants included PTMs such as deamidation, glycation, and one to three N-acetylneuraminic acid residues.

For mAb7, an additional Fab arm was connected to the normal mAb with G4S linkers, and it is referred as “N+1” format. nCE-MS analysis revealed five species, including two basic variants and two acidic variants, as shown in FIG. 16B and Table 7.

TABLE 7
Mass table for charge variant characterization
of mAb7 in “N + 1” format

| Charge Variant Peaks | Proposed Charge Variant Identity |
|---|---|
| Basic 2 | 2× C-terminal Lys |
| Basic 1 | 1× C-terminal Lys |
| Basic 0 | 1× N-terminal pyroglutamate |
| Main | mAb |
| Acidic 1 | Glycation |
| | O-glycan (Xyl + Gal + Gal + GlcA) |
| Acidic 2 | Glycation |

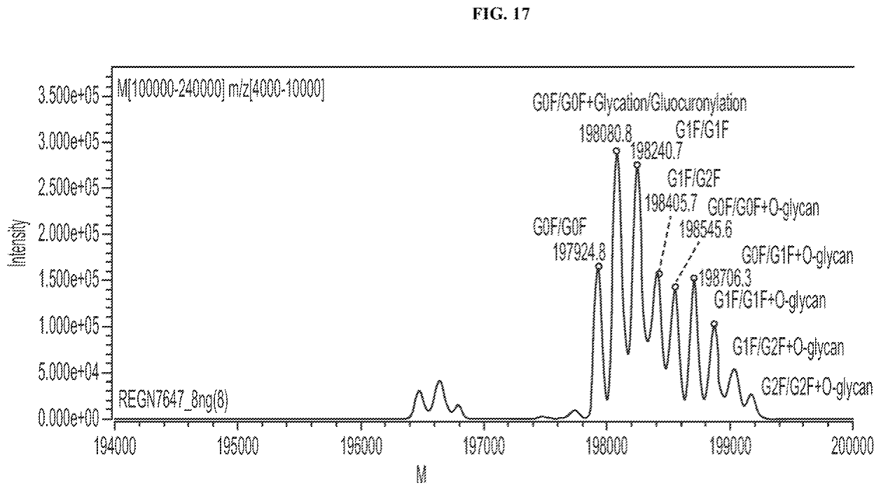

The basic variants were attributed mainly to one or two unprocessed C-terminal Lys residues and N-terminal pyroglutamate formation. In the acidic variants, in addition to commonly observed PTMs such as deamidation and glycation, mass differences matching 0-glycosylation were also detected, as shown in FIG. 17. The 0-glycan has a structure of Xyl+Gal+Gal+GlcA, which is a common 0-glycan in mAb formats using G4S linkers, as reported in previous publications (Haberger et al.; Spahr et al., 2014, MAbs, 6(4):904-914; Spencer et al., 2013, J. Pharm. Sci, 102(11):3920-3924). The presence of O-glycan was confirmed by reduced peptide mapping. In summary, nCE-MS is suitable for the analysis of new formats and can reveal PTMs unique to common mAbs such as O-glycosylation.

### Materials and Methods for Examples 7-10

Chemicals and reagents. Unless stated otherwise, all chemicals and reagents were acquired from MilliporeSigma (Burlington, MA). NISTmAb (reference material 8671, humanized IgG1K monoclonal antibody) was acquired from the National Institute of Standards and Technology (Gaithersburg, MD). MAb8 and bsAb4 were produced internally by Regeneron Pharmaceuticals, Inc. (Tarrytown, NY). Acetonitrile, Invitrogen UltraPure 1 M Tris-HCl buffer, pH 7.5, Tris [2-carboxyethyl] phosphine hydrochloride (TCEP-HCl), and dimethyl sulfoxide (HPLC grade) were acquired from Thermo Fisher Scientific (Waltham, MA). Deionized water (Milli-Q water) was generated with a Milli-Q integral 10 water purification device equipped with a MilliPak Express 20 filter (MilliporeSigma). Sequencing grade modified trypsin and IdeS protease were acquired from Promega Corporation (Madison, WI). ZipChip HRN chips and a native assay kit with Native Antibodies background electrolyte (BGE) were obtained from 908 Devices (Boston, MA).

Sample preparation. For thermal stress studies, NISTmAb was incubated at 45° C., and samples were collected after 15 and 28 days. For subunit analysis, each NISTmAb sample was diluted to 2 mg/mL with Milli-Q water, and 125 U IdeS protease was added to 100 μg of mAb at an enzyme/antibody ratio of 1.25/1. The mixture was incubated at 37° C. with shaking at 600 rpm for 30 minutes to generate F(ab′)2 and Fc fragments. mAb8 was incubated at 25° C. in accelerated stability studies, and one time point at 3 months was collected. The stability of mAb8 under stress was assessed at 45° C., and samples were collected at 3 weeks and 5 weeks. bsAb4 samples were incubated with either 1 ppm H₂O₂ or 10 ppm H₂O₂ at 25° C. for 24 hours. bsAb4 samples subjected to pH stress were prepared at pH values of 5, 6, 7, and 8, and incubated at 40° C. for 28 days. All control and stressed samples were immediately stored in a −20° C. freezer after treatment.

Reduced peptide mapping. For reduced peptide mapping experiments, samples of NISTmAb, mAb8, and bsAb4 were subjected to similar protocols. Briefly, 0.5 mg of each antibody sample was buffer exchanged into 5 mM acetic acid with a Nanosep® 10K centrifugal filter (Pall Corporation, Port Washington, NY). Next, a 0.1 mg aliquot of each sample was denatured and reduced with 5 mM TCEP-HCl at 80° C. for 10 min, diluted with 8 M urea, and subjected to alkylation with 2 mM iodoacetamide and trypsin digestion (enzyme-to-substrate ratio of 1:20 w/w) at 37° C. in the dark for 3 hours. The digested sample was quenched with 5% trifluoracetic acid (TFA).

LC-MS for reduced peptide mapping. The LC-MS conditions were similar for the three antibodies. Experiments were performed with an ACQUITY UPLC Peptide BEH C18 column (1.7 μm, 300 Å, 2.1 mm×150 mm, 1-30 K, Waters Corporation, Milford, MA) on an ACQUITY UPLC I-Class system (Waters Corporation). Mobile phase (MP) A was 0.05% TFA in Milli-Q water, and MPB was prepared with 0.045% TFA in acetonitrile. The gradient differed slightly among the three samples. In general, the gradient was run at 0.25 mL/min, starting with 0.1% MPB from 0 to 5 min, followed by 0.1-35% MPB from 5 to 100 min, 35-90% MPB from 100 to 105 min, 90% MPB from 105 to 110 min, 90%-0.1% MPB from 110 to 111 min, and then 0.1% MPB until the end of the 125 min gradient. The column temperature was set to 40° C. during the experiment. The protein injection amount was 10 μg for all samples.

The LC system was coupled to a Thermo Scientific Q Exactive Plus Hybrid Quadrupole-Orbitrap mass spectrometer (Bremen, Germany). MS data acquisition was performed through a full scan at a resolution of 70,000 in positive mode with a scan range of 300-2,000 m/z, AGC target at 1e6, and maximum injection time of 50 ms. MS/MS experiments were conducted at a resolution of 17,500, AGC at 1e5, maximum injection time of 100 ms, loop count of 5, and normalized collision energy of 27. The ESI parameters were set with a spray voltage of 3.8 kV, capillary temperature of 350° C., S-lens at 50, sheath gas at 40, auxiliary gas at 10, and auxiliary gas heater temperature of 250° C.

Native microfluidic CE-MS. The native microfluidic CE-MS experiment was performed as described above. Briefly, the nCE-MS experiment was conducted with the ZipChip CE interface (908 Devices, Boston, MA) coupled to a Thermo Scientific Exactive Plus EMR Orbitrap mass spectrometer (Bremen, Germany). Samples were loaded on the sample well of a microfluidic chip by a model 840 autosampler. For each experiment, the injection volume for each sample was 1 nL, with a pressure assistance start time at 0.2 min and a replicate delay of 30 seconds between experiments. With the Native Antibodies BGE and 3.8% dimethyl sulfoxide, pH ˜5.5, charge variants of each sample were separated in the microfluidic chip under a field strength of 650 V/cm for 15 min. Each separated charge variant species was subjected to ESI followed by MS analysis. MS data acquisition was performed through a full scan at a resolution of 17,500 in positive mode with a scan range of 1,000-10,000 m/z, in-source CID at 100 eV, AGC at 3e6, maximum injection time of 50 ms, and 3 microscans. The ESI parameters were set to a spray voltage of 0, capillary temperature of 300° C., S-lens at 150, sheath gas at 2, auxiliary gas at 0, and trapping gas at 1.

Data Analysis. Identification of PTMs was performed with Byonic version 4.6.1 (Protein Metric Inc., Cupertino, CA). The relative abundance of PTMs was determined in Skyline-daily software version 21.2.1.424 with selection of all available charge states and three isotopic peaks for both modified and native peptides. For charge variant analysis, electropherograms and intact mass analysis were analyzed with Xcalibur 4.3.73.11 (Thermo Fisher Scientific) and Intact Mass™ version 3.11-1 (Protein Metric Inc.).

### Example 7. Charge Heterogeneity Analysis of NISTmAb Under Stress at the Intact and Subunit Levels

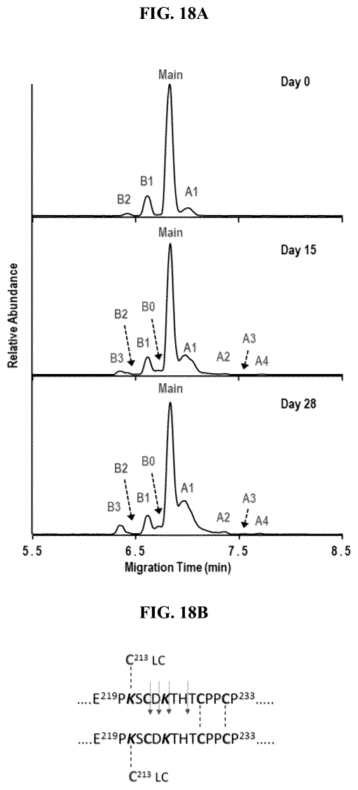

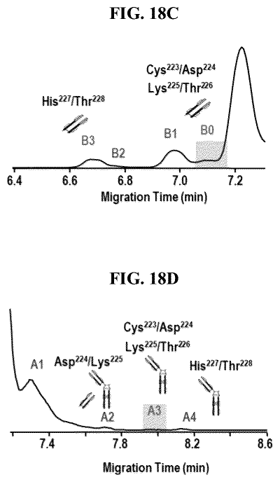

As described above, environmental stress may induce the formation of new charge variants due to both antibody truncations and PTMs. NISTmAb under thermal stress was further analyzed as an example demonstrating these changes. NISTmAb was stressed at 45° C., and samples at two time points (15 and 28 days) were collected. The control and stressed NISTmAb were analyzed with ZipChip nCE-MS, and the electropherograms of intact antibodies are shown in FIG. 18A. In the control sample, the charge variant profile included the basic variants B1 and B2, with 1× and 2× unprocessed C-terminal Lys residues, respectively, and the acidic variant A1, with deamidation and glycation, as shown in Table 8. In the stressed sample on day 15, additional basic variants B0 and B3, and the new acidic variants A2, A3, and A4, were detected. The peak areas for these new basic and acidic variants increased in the stressed sample by day 28. Intact mass analysis revealed the identities of these new variants, which were fragments arising from clipping at the hinge region at four amide bond linkages: Cys223/Asp224, Asp224/Lys225, Lys225/Thr226, and His227/Thr228, as shown in FIG. 18B and Table 8.

TABLE 8
Charge variant identities for control and stressed NISTmAb

| Charge | Control | |
|---|---|---|
| Variants | NISTmAb | Stressed NISTmAb (28 days) |
| Basic 3 (B3) | Not detected | One Fab arm cleaved at His²²⁷/Thr²²⁸ |
| Basic 2 (B2) | +2× C-terminal | +2× C-terminal lysine |
| | lysine | |
| Basic 1 (B1) | +1× C-terminal | +1× C-terminal lysine |
| | lysine | |
| Basic 0 (B0) | Not detected | One Fab arm cleaved at |
| | | Cys²²³/Asp²²⁴ and |
| | | Lys²²⁵/Thr²²⁶ |
| Acidic 1 (A1) | +Deamidation | +Deamidation |
| Acidic 2 (A2) | Not detected | Fab cleavage at Asp²²⁴/Lys²²⁵ |
| Acidic 3 (A3) | Not detected | Loss of one Fab arm cleaved at |
| | | Cys²²³/Asp²²⁴ and |
| | | Lys²²⁵/Thr²²⁶ |
| Acidic 4 (A4) | Not detected | Loss of one Fab arm cleaved at |
| | | His²²⁷/Thr²²⁸ |

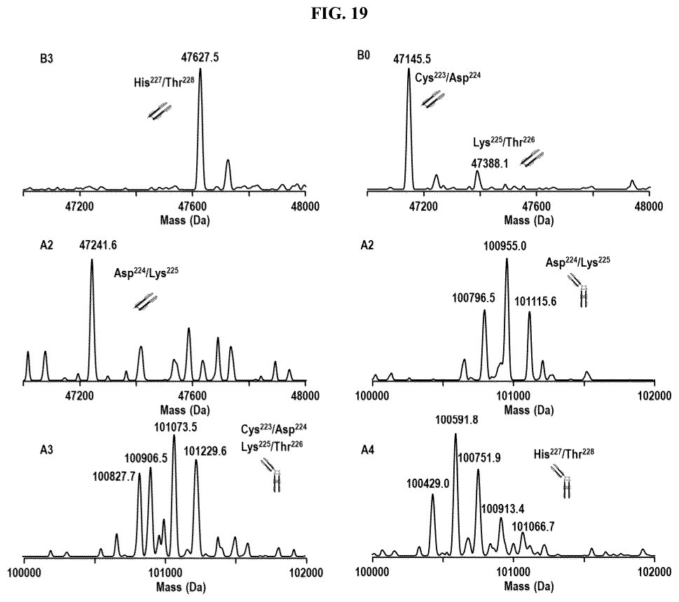

Both the Fab arm and the remainder of the mAb were detected by MS: the Fab arms were detected in B0, B3, and A2, and the remainders of the mAb were observed in A2, A3, and A4, as shown in FIG. 18C, FIG. 18D, and FIG. 19. Additionally, nCE-MS analysis indicated that the peak area of the A1 variant increased over time.

The ZipChip nCE-MS method results were subsequently compared with those from the reduced peptide mapping analysis. The reduced peptide mapping of the NISTmAb thermal stressed revealed an increase in Asn deamidation at HC Asn387 and Asn392, Met oxidation at HC Met255, and Asp isomerization at HC Asp283, as shown in Table 9. Because deamidation was the main contributor to the formation of the A1 variant, this observation was consistent with the data from the reduced peptide mapping. However, in the case of NISTmAb, Met oxidation and Asp isomerization was not confidently identified in the basic variants through ZipChip nCE-MS analysis, possibly because of the relatively low level of both Met oxidation and Asp isomerization in comparison to the high abundance of other basic variants. In contrast, reduced peptide mapping had limitations in the analysis of sequence truncation, whereas this information was successfully revealed by ZipChip nCE-MS analysis.

TABLE 9
PTM analysis of NISTmAb under thermal stress
conditions by reduced peptide mapping

| Antibody | | | D 0 | | |
|---|---|---|---|---|---|
| Region | PTMs | Site | (Control) | D 15 | D 28 |
| Fc | Deamidation | HC Asn³⁸⁷ | 1.6% | 2.1% | 2.6% |
| | Deamidation | HC Asn³⁹² | 0.8% | 2.0% | 3.0% |
| | Oxidation | HC Met²⁵⁵ | 1.2% | 2.1% | 3.0% |
| | Isomerization | HC Asp²⁸³ | 0.7% | 1.7% | 2.8% |

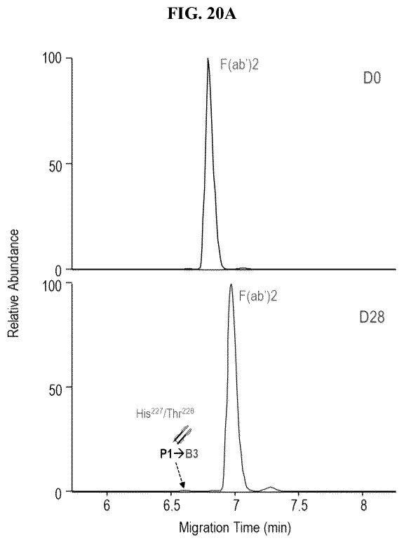

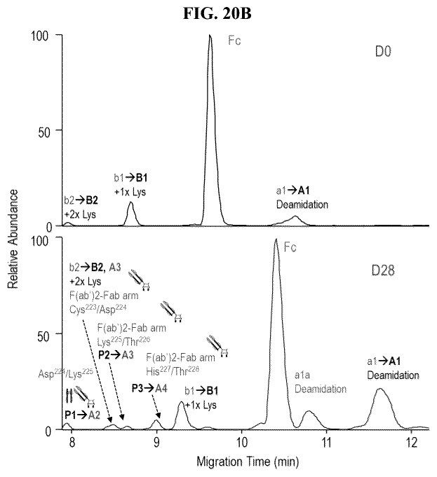

Subsequently, the stressed samples were analyzed at the subunit level, after being digested by IdeS to generate F(ab′)2 and Fc subunits. As shown in FIG. 20A and FIG. 20B, F(ab′)2 and Fc were well separated by the universal CE-MS method; in the same run, F(ab′)2 and Fc were observed at approximately 7 and 10 minutes, respectively. All identified minor peaks are shown in FIG. 20A and FIG. 20B, and new charge variants resulting from the 28-day incubation matched those identified through intact NISTmAb analysis. In the F(ab′)2 region, Fab arm clipping at His227/Thr228 was observed and corresponded to B3 identified in the intact NISTmAb analysis, as shown in FIG. 20A. In the Fc region, masses matching F(ab′)2—Fab arm clipping at Cys223/Asp224, Asp224/Lys225, Lys225/Thr226, and His227/Thr228 were identified, and these peaks corresponded to the new charge variants in A2, A3, and A4 in the intact NISTmAb analysis of samples at 28 days, as shown in FIG. 20B.

The Fab cleavage sites were found in the basic region of F(ab′)2, in the same order as those identified in the acidic region during intact NISTmAb analysis. For the Fc acidic region, the new acidic variant ala appeared in the D28 sample, as shown in FIG. 20B. With respect to the main Fc, the +1 Da mass increase in both al and ala indicated that the acidic variant might have been caused by deamidation, as indicated by the reduced peptide mapping analysis, as shown in Table 9.

Comparison of intact and subunit analyses of NISTmAb stressed samples with ZipChip nCE-MS revealed the same charge variants. Subunit analysis provided an advantage in the confidence of identification because higher mass accuracy was more easily obtainable due to the lower molecular weight of antibody subunits compared to intact antibodies. Identification of PTMs such as deamidation, succinimide, and glycation with small mass errors has been demonstrated through subunit analysis (Madren and Yi, 2022, Electrophoresis, 43(23-24):2453-2465). Additionally, the PTMs or fragments observed in charge variants could be localized to the antibody subunits. In contrast, intact nCE-MS analysis includes most of the information provided in subunit nCE-MS analysis, and can be further used for comparison with other methods for analyzing intact antibodies, such as capillary isoelectric focusing and cation exchange chromatography (Sun et al.; Fussl et al.).

In summary, in analysis of NISTmAb under thermal stress conditions, the ZipChip nCE-MS method of the present invention provided a highly sensitive analytical method for detection of charge variants from deamidation and antibody fragments arising from clipping at the hinge region, in both intact and subunit analyses.

### Example 8. Changes in Charge Variants Due to Alterations in Post-Translational Modification Levels Under Thermal Stress Conditions

Beyond the truncated species appearing as new charge variants in the case of NISTmAb, changes in PTM levels also resulted in the formation of new charge variants, as illustrated in a case study of mAb8 under thermal stress. In this study, mAb8 was subjected to accelerated stability conditions (25° C.) and thermal stress conditions (45° C.), and changes in charge variants were analyzed with ZipChip nCE-MS. In a control sample, the basic charge variants of mAb8 were composed of 1× and 2× unprocessed C-terminal Lys, 1× and 2× glycine loss with amidation, and 1× or 2× pyroglutamate species, whereas the acidic charge variants were composed of glycated, deamidated, and sialylated species.

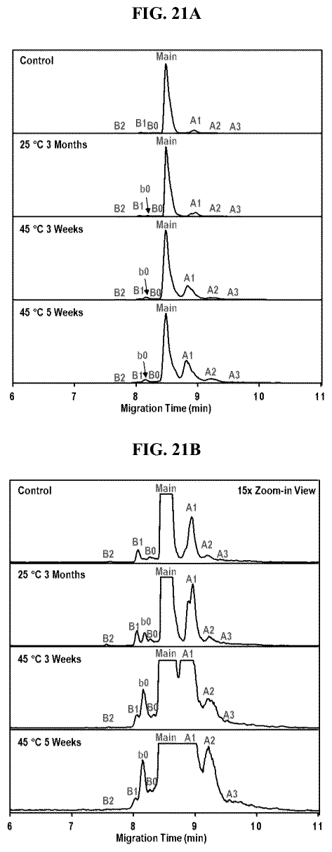

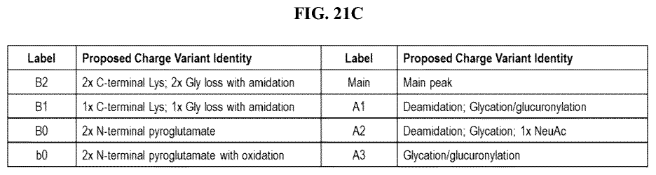

The migration order of the basic variants containing unprocessed C-terminal Lys, N-terminal pyroglutamate, and main species was consistent with that previously reported (Carillo et al., 2020, J Pharm Biomed Anal, 185:113218). Under accelerated stability and thermal stress conditions, a new basic variant, b0, was observed in both accelerated stability and thermal stress samples, as shown in FIG. 21A and FIG. 21B. The b0 peak area increased over time from the 3-week time point to the 5-week time point. PTM identifications proposed for the observed charge variants are shown in FIG. 21C.

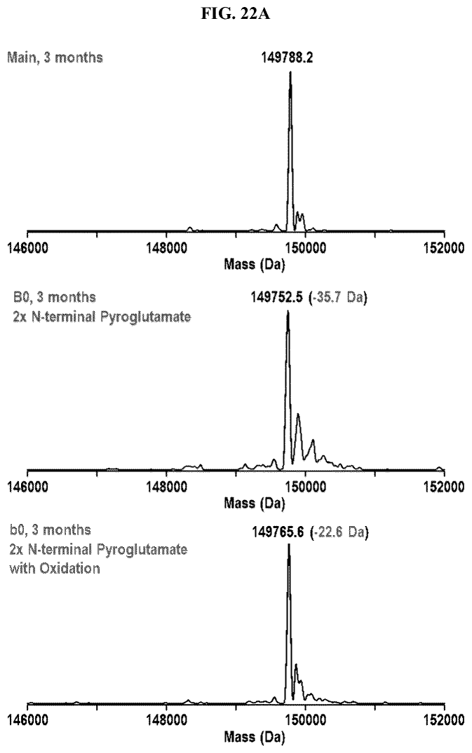

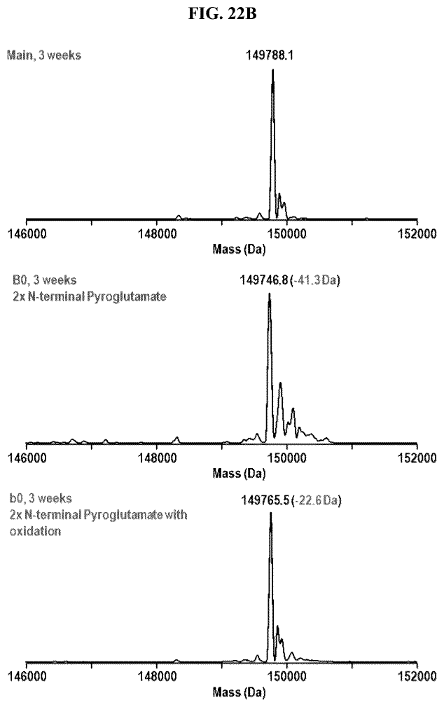

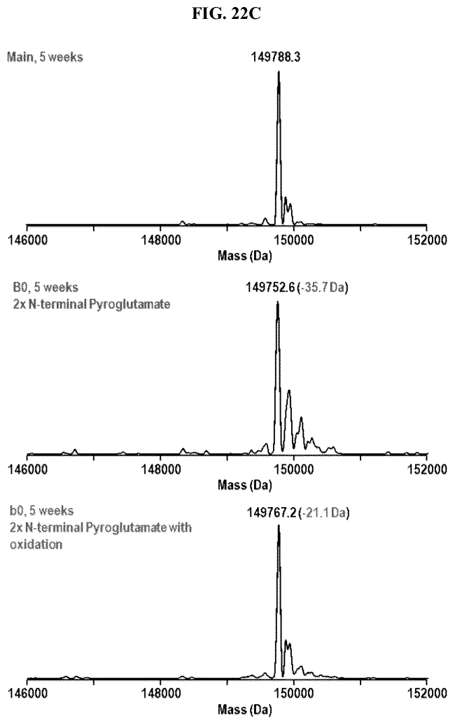

MS analysis was used to examine the mass difference between B0 and b0. Compared with the main species, the basic variant B0 had a mass shift of approximately −36 Da, which was annotated as 2× N-terminal pyroglutamate, as shown in FIG. 22A, FIG. 22B and FIG. 22C. In comparison, the basic variant b0 had a mass shift of approximately −22 Da, which could have arisen from a combination of 2× N-terminal pyroglutamate (−36 Da) with oxidation (+16 Da). Similar to the results of NISTmAb analysis, the peak area of acidic variant A1 increased, probably because the thermal stress condition caused higher deamidation levels.

To validate the proposed PTM assignments of the charge variant species, reduced peptide mapping analysis was used to examine the changes in PTM levels in stressed mAb8. Under either accelerated stability or thermal stress conditions, the oxidation levels at two HC oxidation hotspots at CH2 and the CH2-CH3 interface, the N-terminal pyroglutamate formation level, and the deamidation level significantly increased, as shown in Table 10. All other amino acid residues did not show noticeable changes in PTM levels. The results of the reduced peptide mapping analysis were consistent with the observations from ZipChip nCE-MS analysis, wherein charge variant identities were deduced according to differences in the observed masses between the charge variants and the main peak. The ZipChip nCE-MS analysis of mAb8 under accelerated stability and thermal stress conditions successfully enabled PTM annotations to be deduced for both N-terminal pyroglutamination and oxidation in the charge variant, on the basis of intact mass analysis.

TABLE 10
PTM analysis of mAb8 under accelerated
stability and thermal stress conditions

| | | | 3 months | 3 weeks | 5 weeks |
|---|---|---|---|---|---|
| PTMs | Site | Control | (25° C.) | (45° C.) | (45° C.) |
| Deamidation | HC Asn³³⁰ | 0.2% | 1.1% | 7.2% | 10.8% |
| | HC Asn³⁸⁹ | 1.7% | 2.1% | 3.3% | 4.5% |
| Methionine | HC Met²⁵⁷ | 5.8% | 9.4% | 11.4% | 14.7% |
| Oxidation | HC Met⁴³³ | 1.0% | 2.2% | 3.0% | 4.1% |
| N-terminal | HC Glu¹ | 1.9% | 2.7% | 5.2% | 7.3% |
| Pyroglutamate | | | | | |

### Example 9. Charge Variant Changes Under Oxidative Stress Conditions

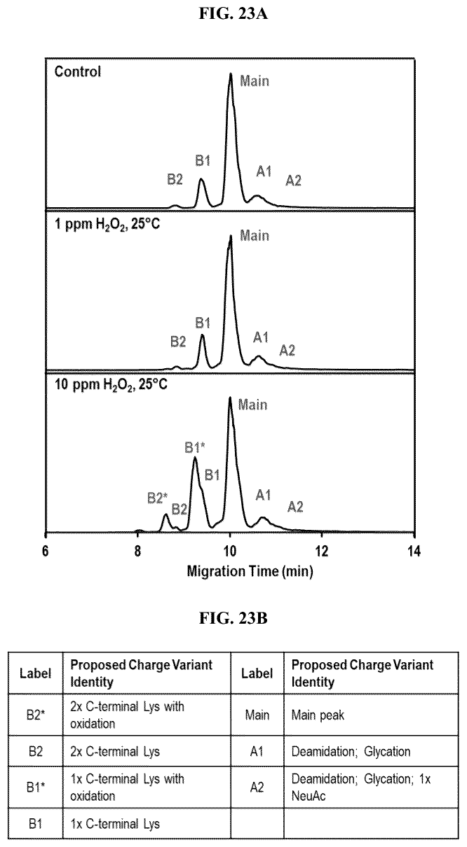

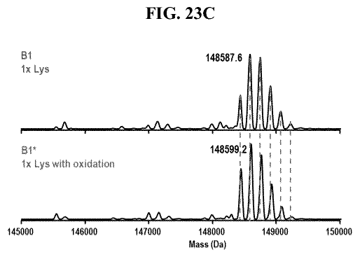

In addition to thermal stress conditions, other stress conditions, such as pH and oxidative stresses, can alter charge variant profiles. To investigate whether ZipChip nCE-MS might be used to analyze charge variants in these stress conditions, bsAb4 was subjected to oxidative stress with H₂O₂. In this stress condition, oxidation is the major contributor to charge variant changes (Liu et al., 2008, Biochemistry, 47(18):5088-5100). In the control sample, 1× and 2× unprocessed C-terminal Lys residues were identified in the basic variants B1 and B2, whereas deamidation, glycation, and 1×NeuAc were identified in the acidic variants A1 and A2, as shown in FIG. 23A and FIG. 23B. Incubation of bsAb4 with 1 ppm H₂O₂ at 25° C. did not result in major differences in the charge variant profile. However, the new charge variants B1* and B2* were observed under oxidative stress conditions at 10 ppm H₂O₂. On the basis of intact mass analysis, the basic variants B1* and B2* were identified as the oxidized species of the basic variants B1 and B2, respectively. As shown in FIG. 23C, a mass increase close to oxidation (+16 Da) was observed in all glycoforms from basic variant B1 to basic variant B1*. The proposed charge variant identifications were supported by reduced peptide mapping analysis, which indicated a modest increase in oxidation levels in the two oxidation sites at 1 ppm H₂O₂, as well as a large increase in the oxidation level at 10 ppm H₂O₂, as shown in Table 11. Whereas oxidized species were separated in the basic variant B1 and B2, oxidized species were not observed in the main and acidic species, possibly because of the less optimal separation at later migration times.

TABLE 11
PTM analysis of bsAb4 under oxidative stress conditions

| PTMs | Site | Control | 1 ppm H₂O₂ | 10 ppm H₂O₂ |
|---|---|---|---|---|
| Oxidation | HC Met²⁵⁶ | 3.5% | 4.8% | 15.8% |
| | HC* Met²⁵⁵ | | | |
| | HC Met³⁶² | 0.5% | 0.8% | 2.9% |
| | HC* Met³⁶¹ | | | |

### Example 10. Charge Variant Changes Under pH Stress Conditions

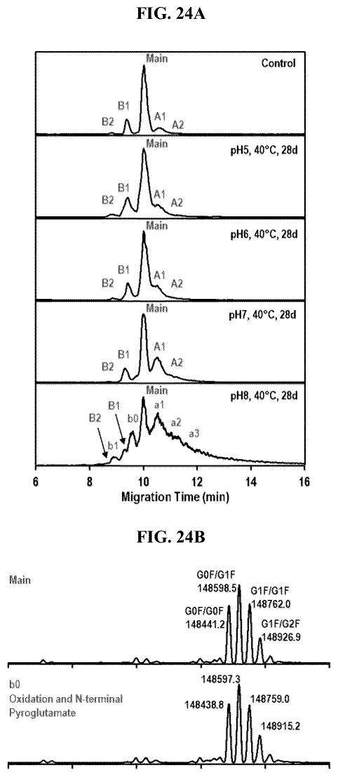

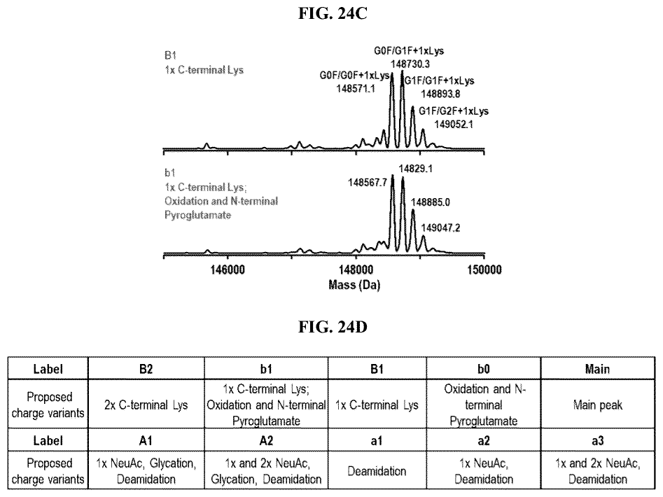

The method of the present invention was further used to investigate charge variants arising from pH stress. bsAb4 was incubated at pH 5, 6, 7, or 8 for 28 days at 40° C. In the control sample, bsAb4 showed two basic variants with PTMs including 1× and 2× unprocessed Lys residues in B1 and B2, respectively, and two acidic variants with PTMs such as deamidation, glycation, and sialylation, as shown in FIG. 24A. Compared with the control sample, the pH stressed samples at pH 5, 6, and 7 did not show appreciable changes in the charge variant profiles, even though the incubation was performed under an elevated temperature of 40° C. At pH 8, the new charge variants b0 and b1 were observed. Intact mass analysis revealed a similar glycan distribution and masses between the main species and b0, as shown in FIG. 24B, and similar findings were observed in B1 and b1, as shown in FIG. 24C. Because the mass changes for oxidation and N-terminal pyroglutamination are +16 Da and −18 Da, respectively, the combined effects of these two PTMs would generate peaks with small mass differences between species in the deconvoluted mass spectra. Additionally, the clear increase in acidic species observed in the electropherogram suggested that deamidation markedly increased, as shown in FIG. 24D.

Reduced peptide mapping analysis demonstrated an increase in oxidation and N-terminal pyroglutamination at selected amino acid residues, as shown in Table 12. The considerable increase in deamidation level was confirmed by reduced peptide mapping analysis, which indicated an increase in the deamidation level of the PENNYK peptide, a deamidation hotspot, from 2.4% in the control to 11.1% and 46.1% for samples subjected to stress at pH 7 and pH 8, respectively. These observations were also consistent with the findings from ZipChip nCE-MS and other reports in which higher pH caused higher levels of deamidation (Pace et al., 2013, J Pharm Sci, 102(6):1712-1723).

TABLE 12
PTM analysis of bsAb4 under pH stress conditions

| PTMs | Site | Control | pH 5 | pH 6 | pH 7 | pH 8 |
|---|---|---|---|---|---|---|
| Deamidation | HC Asn³⁹³ | 2.4% | 3.0% | 4.6% | 11.1% | 46.1% |
| | HC* Asn³⁹² | | | | | |
| Methionine | HC Met²⁵⁶ | 3.5% | 5.5% | 5.9% | 8.8% | 12.2% |
| Oxidation | HC* Met²⁵⁵ | | | | | |
| N-terminal | HC Glu¹ | 2.3% | 3.4% | 4.1% | 10.4% | 27.9% |
| Pyroglutamate | HC* Glu¹ | 1.9% | 3.8% | 3.1% | 8.3% | 22.0% |

ZipChip nCE-MS enables fast charge variant analysis through intact mass analysis with minimal sample preparation. In this disclosure, characterization of charge variants and impurities of therapeutic antibodies was demonstrated with nCE-MS method, which provides high resolution separation and high sensitivity detection, with excellent system robustness. The electropherograms provided by nCE-MS analysis were similar to the chromatograms from SCX and the electropherograms from iCIEF. Therefore, this method can be used for peak identification in SCX and iCIEF studies. Antibodies with close pI values can be separated well. Co-migrated antibodies were identified individually based on simplified native mass spectra. Increased levels of acidic variants and Fab fragments resulting from incubation under stressed conditions were localized within the F(ab′)2 and Fc domains by subunit analysis. Furthermore, higher resolution subunit analysis revealed an additional acid variant introduced from isomerization and increased half glycosylated species under stressed condition. Moreover, high sensitivity detection enables the detection of impurities including truncations in NISTmAb and homospecific mAb impurities in bsAb. In addition to common therapeutic antibodies, nCE-MS analysis can be applied to characterize new modalities including mAbs with or without Fab arms and to detect PTMs such as O-glycosylation.

Additionally, characterization of charge variants under various stress and stability conditions was demonstrated with the ZipChip nCE-MS method. Under thermal stress conditions, both deamidation and sequence truncations at the hinge region were responsible for the changes observed in the charge variant profile of NISTmAb. In the case of mAb8 under thermal stress and stability conditions, the changes in the charge variant profile were associated primarily with PTMs including deamidation, pyroglutamination, and oxidation. For bsAb4 under oxidative stress, ZipChip nCE-MS was able to separate the charge variants associated with oxidation. For bsAb4 under pH stress, deamidation, pyroglutamination, and oxidation contributed to the new charge variants observed in the electropherogram. In summary, ZipChip nCE-MS can aid in process development of biotherapeutics by elucidating the new charge variants arising under various stress and stability conditions.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for identifying, quantifying, and/or characterizing at least one charge variant of a protein of interest, comprising:
(a) subjecting a sample including a protein of interest to stress conditions to produce a sample including at least one charge variant of said protein of interest;
(b) subjecting said sample including at least one charge variant of said protein of interest to native capillary electrophoresis to produce an eluate; and
(c) subjecting said eluate to mass spectrometry analysis to identify, quantify, and/or characterize said at least one charge variant of said protein of interest,
wherein said native capillary electrophoresis is performed in an integrated microfluidic device directly coupled to said mass spectrometer.

2. The method of claim 1, wherein said protein of interest is selected from a group consisting of an antibody, a monoclonal antibody, a monospecific antibody, a bispecific antibody, a multispecific antibody, an antibody fusion protein, an antibody-drug conjugate, an antibody fragment, an antibody subunit, an antigen-binding protein, and antibody-derived protein, and a therapeutic antibody.

3. The method of claim 1, wherein a duration of said stress conditions is from 0 hours to 24 hours, from 0 days to 30 days, from 0 weeks to 8 weeks, from 0 months to 12 months, about 1 hour, about 6 hours, about 12 hours, about 18 hours, about 24 hours, about 1 day, about 2 days, about 3 days, about 4 days, about 5 days, about 6 days, about 7 days, about 14 days, about 15 days, about 21 days, about 28 days, about 1 week, about 2 weeks, about 3 weeks, about 4 weeks, about 5 weeks, about 6 weeks, about 7 weeks, about 8 weeks, about 1 month, about 2 months, about 3 months, about 4 months, about 5 months, about 6 months, about 7 months, about 8 months, about 9 months, about 10 months, about 11 months, or about 12 months.

4. The method of claim 3, wherein said stress conditions comprise thermal stress, pH stress, oxidation stress, ultraviolet stress, and combinations thereof.

5. The method of claim 4, wherein said thermal stress comprises subjecting said sample to a temperature from 20° C. to 80° C., from 25° C. to 50° C., about 25° C., about 30° C., about 35° C., about 37° C., about 40° C., about 45° C., about 50° C., about 55° C., about 60° C., about 65° C., about 70° C., about 75° C., or about 80° C.

6. The method of claim 4, wherein said pH stress comprises subjecting said sample to a pH from 4 to 10, about 4, about 5, about 6, about 7, about 8, about 9, or about 10.

7. The method of claim 4, wherein said oxidation stress comprises contacting said sample to H₂O₂ at a concentration from 0.5 ppm to 20 ppm, about 0.5 ppm, about 1 ppm, about 2 ppm, about 3 ppm, about 4 ppm, about 5 ppm, about 6 ppm, about 7 ppm, about 8 ppm, about 9 ppm, about 10 ppm, about 15 ppm, or about 20 ppm.

8. The method of claim 1, wherein said at least one charge variant comprises an acidic variant and/or a basic variant.

9. The method of claim 1, further comprising attributing said at least one charge variant to at least one post-translational modification.

10. The method of claim 9, wherein said at least one post-translational modification comprises deamidation, oxidation, glycation, disulfide formation, N-terminal pyroglutamate formation, C-terminal lysine removal, high mannose glycosylation, O-glycosylation, isomerization, truncation, or combinations thereof.

11. The method of claim 1, further comprising repeating steps (a)-(c) using at least one additional stress condition or control condition.

12. The method of claim 1, wherein an injection volume of said sample for said native capillary electrophoresis is from 0.5 nL to 2 nL, about 0.5 nL, about 1 nL, about 1.5 nL, or about 2 nL.

13. The method of claim 1, wherein said integrated microfluidic device comprises an integrated electrospray ionization emitter.

14. The method of claim 1, further comprising subjecting said sample including said at least one charge variant of said protein of interest to digestion conditions prior to step (b).

15. The method of claim 14, wherein said digestion conditions comprise contacting said sample to at least one digestive enzyme selected from a group consisting of pepsin, trypsin, Tryp-N, chymotrypsin, Lys-N, Lys-C, Asp-N, Arg-C, Glu-C, papain, IdeS, or a variant thereof.


SEQUENCE LISTING (1 sequence)

SEQ ID NO: 1 - protein, 14 aa, synthetic construct
  REGION 1..14: note = Enzymatically produced peptide
PKSCDKTHTCPPCP